(19) United States
(12) Patent Application Publication
(10) Pub. No.: US 2024/0139170 A1
(43) Pub. Date: May 2, 2024

(54) TOPICAL FORMULATIONS OF A JAK 1/3 INHIBITOR AND METHODS OF USE THEREOF FOR TREATMENT OF ATOPIC DERMATITIS AND OTHER SKIN CONDITIONS

(71) Applicant: ACLARIS THERAPEUTICS, INC., Wayne, PA (US)

(72) Inventors: Paul CHANGELIAN, Framingham, MA (US); David GORDON, Ardmore, PA (US); Francis IGNATIOUS, Exton, PA (US); Walter SMITH, St. Louis, MO (US); Steve TUCKER, Wallingford, PA (US); Neal Stuart WALKER, Phoenixville, PA (US)

(21) Appl. No.: 18/277,886

(22) PCT Filed: Feb. 18, 2022
(86) PCT No.: PCT/US2022/070734

§ 371 Date: Aug. 18, 2023

Related U.S. Application Data
(60) Provisional application No. 63/221,706, filed on Jul. 14, 2021.
(60) Provisional application No. 63/197,888, filed on Jun. 7, 2021.
(60) Provisional application No. 63/197,463, filed on Jun. 6, 2021.
(60) Provisional application No. 63/150,610, filed on Feb. 18, 2021.

(51) Int. Cl. A61K 31/4545; A61K 9/00; A61K 9/08; A61K 45/06; A61K 47/10; A61K 47/20; A61K 47/22; A61K 47/34; A61P 17/00; A61P 17/02

(52) U.S. Cl. CPC A61K 31/4545; A61K 9/0014; A61K 9/08; A61K 45/06; A61K 47/10; A61K 47/20; A61K 47/22; A61K 47/34; A61P 17/00; A61P 17/02

(57) ABSTRACT

The present disclosure relates methods for treating skin disorders caused by an immune deficiency with topical formulations comprising ethyl (R)-4-((1-(2-cyanoacetyl)piperidin-3-yl)amino)-1H-pyrrolo[2,3-b]pyridine-5-carboxylate (Compound I) or pharmaceutically acceptable derivative thereof. In particular, the topical formulations described herein may be used to treat atopic dermatitis, alopecia areata, or vitiligo.

### CLAIM OF PRIORITY

This application claims the benefit of priority to U.S. Provisional Application No. 63/150,610, filed Feb. 18, 2021, U.S. Provisional Application No. 63/197,463, filed Jun. 6, 2021, U.S. Provisional Application No. 63/197,888, filed Jun. 7, 2021, and U.S. Provisional Application No. 63/221,706, filed Jul. 14, 2021, which are incorporated herein by reference in their entirety.

### FIELD

The present disclosure is directed to the preparation and use of topical formulations comprising a JAK inhibitor and the use of those formulations for treating a skin condition, in particular, for treating atopic dermatitis.

### SUMMARY OF THE DISCLOSURE

A first aspect of the disclosure is directed to a method for treating a skin condition caused by immune deficiency in a human subject in need thereof. This method comprises administering topically to a region of the subject affected by the skin condition, a composition comprising about 0.5% to about 4% of ethyl (R)-4-((1-(2-cyanoacetyl)piperidin-3-yl)amino)-1H-pyrrolo[2,3-b]pyridine-5-carboxylate having the structure of Compound I or a salt thereof

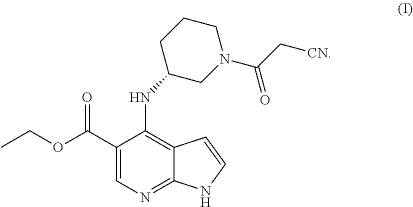 [SMILES: CCOC(=O)c1cnc2[nH]ccc2c1N[C@@H]1CCCN(C(=O)CC#N)C1]

Another aspect of the disclosure is directed to a method for reducing severity and extent of skin lesions caused by an immune deficiency. This method comprises administering topically, to a human subject having the immune deficiency, a composition comprising about 0.5% to about 4% of ethyl (R)-4-((1-(2-cyanoacetyl)piperidin-3-yl)amino)-1H-pyrrolo[2,3-b]pyridine-5-carboxylate having the structure of Compound I (above) or a salt thereof

Another aspect of the disclosure is directed to a topical pharmaceutical formulation comprising about 0.1 w/w % to about 4 w/w % of ethyl (R)-4-((1-(2-cyanoacetyl)piperidin-3-yl)amino)-1H-pyrrolo[2,3-b]pyridine-5-carboxylate (Compound I), a salt thereof, or a solvate thereof. The formulation further comprises a primary solvent that comprises one or both of a polyethylene glycol having an average molecular weight of about 100 g/mol to about 400 g/mol and diethylene glycol monoethyl ether; a co-solvent comprising one or more of benzyl alcohol, ethanol, and phenoxyethanol, dimethyl isosorbide; propylene glycol; glycerol; and an antioxidant. The topical pharmaceutical formulation of the present disclosure has an apparent viscosity of about 10 mPa·s to about 100 mPa·s.

In yet another aspect, the present disclosure is directed to a sprayable topical pharmaceutical formulation. The sprayable topical formulation comprises about 0.5 w/w % to about 2 w/w % of a compound of the structure of Compound I, a salt thereof, or a solvate thereof; about 65 w/w % to about 70 w/w % of a primary solvent comprising one or both of a polyethylene glycol having an average molecular weight of about 100 g/mol to about 400 g/mol and diethylene glycol monoethyl ether; about 2 w/w % of benzyl alcohol; about 15 w/w % of dimethyl isosorbide; about 5 w/w % of propylene glycol; about 8 w/w % to about 10 w/w % of glycerol; and about 0.05 w/w % to about 0.5 w/w % of an antioxidant, where the topical pharmaceutical formulation has an apparent viscosity of about 10 mPa·s to about 100 mPa·s.

Topical delivery, especially in treating skin conditions such as AD as described herein has a number of advantages: the ability to deliver drug substance more selectively to a specific site, avoiding fluctuations in drug levels, inter- and intra-patient variations, improved compliance, and an enhanced suitability for self-medication. However, many of the current topical therapies for AD and other inflammatory skin conditions (e.g., alopecia areata, vitiligo) face challenges such as imperfect efficacy, difficulty with application, adverse effects with long-term topical use (especially steroidal treatments), and local site reactions. Regarding topical therapies, the formulation of the vehicle is as important as the active agent itself because the interaction of the vehicle with the skin and the active ingredient can alter the efficacy of the active ingredient. In addition to performing adequate studies on the therapeutic efficacy of JAK inhibitors to treat inflammatory skin conditions, adequate formulation development must also be undertaken to ensure that the JAK inhibitor molecule is delivered to the right target site (e.g., epidermis/dermis, avoiding systemic introduction) and that it maintains dosage integrity, drug transport, and active duration, and not the least, patient acceptability and compliance.

As described herein, ethyl (R)-4-((1-(2-cyanoacetyl)piperidin-3-yl)amino)-1H-pyrrolo[2,3-b]pyridine-5-carboxylate (Compound I) was designed to act locally in the skin and to be metabolically labile, thereby resulting in short-lived circulating drug levels in the blood plasma that provide efficacy and minimize the risks of systemic toxicity. When administered to human subjects suffering from moderate to severe atopic dermatitis for a 4-week period, an analysis of pharmacokinetic plasma samples in the treatment arm showed greater than 86% of the plasma samples had concentrations of the active compound that were below 1 ng/ml and mean drug levels were never greater than 5% of the IC₅₀ of ethyl (R)-4-((1-(2-cyanoacetyl)piperidin-3-yl)amino)-1H-pyrrolo[2,3-b]pyridine-5-carboxylate. Subjects treated with ethyl (R)-4-((1-(2-cyanoacetyl)piperidin-3-yl)amino)-1H-pyrrolo[2,3-b]pyridine-5-carboxylate reported a 74.4% reduction in the modified Eczema Area and Severity Index (mEASI) score at week 4, which was the primary efficacy endpoint. In addition, positive trends in key secondary efficacy endpoints were also observed.

### BRIEF DESCRIPTION OF THE DRAWINGS

For a fuller understanding of the nature and advantages of the present invention, reference should be made to the following detailed description taken in connection with the accompanying drawings.

FIG. 1 shows the percent change in Eczema Area and Severity Index (EASI) from baseline for the Compound I and vehicle treated subjects at days 8, 15 and 28.

FIG. 2 shows the EASI 50, EASI 75, and EASI 90 responder data for Compound I and vehicle treated subjects at day 28 of the study.

FIG. 3 shows the change in Peak Pruritus Numerical Rating Scale (PP-NRS) score from baseline in both Compound I and vehicle treated subjects at days 8, 15, and 28 of the study.

FIG. 4 shows the proportion of subjects (Compound I and vehicle treated) having an Investigator's Global Assessment (IGA) treatment response of 0 or 1 with ≥2 point improvement during the study.

FIG. 5 shows the change in body surface area (BSA) impacted by the disease in Compound I and vehicle treated subjects from baseline at each of days 8, 15 and 28.

FIG. 6 is a table summarizing the number of patients achieving 50% improvement in their Eczema Area and Severity Index (EASI) score at days 8, 15, and 28 of the study.

FIG. 7 is a table summarizing the number of patients achieving 75% improvement in their EASI score at days 8, 15, and 28 of the study.

FIG. 8 is a table summarizing the number of patients achieving 90% improvement in their EASI score at days 8, 15, and 28 of the study.

FIG. 9 is a table summarizing the actual values and change from baseline in the Peak Pruritus Numerical Rating Scale (PP-NRS) for Compound I and vehicle treated subjects over the course of the study.

FIGS. 10A-10B are graphs showing the permeation of various formulations described herein into the dermis and epidermis in an ex vivo/in vitro permeability assay as described herein.

FIGS. 11A-11B are graphs showing the permeation of Compound I containing formulations of the disclosure as measured in the in vitro/ex vivo permeability assays described herein.

### DETAILED DESCRIPTION

### Definitions

Before the present compositions and methods are described, it is to be understood that the scope of the invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference with respect to the aspect it is identified as describing. Nothing herein is to be construed as an admission that the invention is not entitled to antedate such disclosure by virtue of prior invention.

It must also be noted that as used herein and in the appended claims, the singular forms “a,” “an,” and “the” include plural reference unless the context clearly dictates otherwise. Thus, for example, reference to a “JAK inhibitor” is a reference to one or more JAK inhibitors and equivalents thereof known to those skilled in the art, and so forth.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.” The compositions and methods of the present disclosure can comprise, consist essentially of, or consist of, the components disclosed.

As used herein, the term “about” means plus or minus 10% of the numerical value of the number with which it is being used. Therefore, about 50% means in the range of 45%-55%.

“Administering” when used in conjunction with a therapeutic means to administer a therapeutic to a patient whereby the therapeutic positively impacts the tissue to which it is targeted. Thus, as used herein, the term “administering”, when used in conjunction with a JAK inhibitor compound described herein, can include, but is not limited to, providing a JAK inhibitor compound into or onto the target tissue.

As used herein, the term “a derivative thereof” refers to a salt thereof, a pharmaceutically acceptable salt thereof, an ester thereof, a free base form thereof, a solvate thereof, a deuterated derivative thereof, a hydrate thereof, an N-oxide thereof, a clathrate thereof, a prodrug thereof, a polymorph thereof, a stereoisomer thereof, a geometric isomer thereof, a tautomer thereof, a mixture of tautomers thereof, an enantiomer thereof, a diastereomer thereof, a racemate thereof, a mixture of stereoisomers thereof, an isotope thereof (e.g., tritium, deuterium), or a combination thereof.

Compounds described herein may contain an asymmetric center and may thus exist as enantiomers. Where the compounds according to embodiments herein possess two or more asymmetric centers, they may additionally exist as diastereomers. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

Conventional techniques for the preparation/isolation of individual enantiomers include chiral synthesis from a suitable optically pure precursor or resolution of the racemate using, for example, chiral high pressure liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art. Chiral compounds of embodiments herein (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Stereoisomer conglomerates may be separated by conventional techniques known to those skilled in the art. See, e.g. “Stereochemistry of Organic Compounds” by Ernest L. Eliel (Wiley, New York, 1994).

The term “substantially free” as used herein, alone or in combination, refers to the absence of isomers within the limits of detection of analytical methods such as nuclear magnetic resonance (NMR), gas chromatography/mass spectroscopy (GC/MS), high performance liquid chromatography (HPLC), or liquid chromatography/mass spectroscopy (LC/MS).

The term “condition” as used herein is intended to be generally synonymous, and is used interchangeably with, the terms “disorder,” “syndrome,” and “disease”, in that all reflect an abnormal condition of the human or animal body or of one of its parts that impairs normal functioning, is typically manifested by distinguishing signs and symptoms, and causes the human or animal to have a reduced duration or quality of life.

As used herein, the term “pharmaceutically acceptable salt” refers to a salt prepared from a base or acid which is acceptable for administration to a patient, such as a mammal. The term “pharmaceutically acceptable salts” embraces salts commonly used to form alkali metal salts and to form addition salts of free acids or free bases. The nature of the salt is not critical, provided that it is pharmaceutically-acceptable. Such salts can be derived from pharmaceutically-acceptable inorganic or organic bases and from pharmaceutically-acceptable inorganic or organic acids.

Suitable pharmaceutically acceptable acid addition salts of the compounds of embodiments herein may be prepared from an inorganic acid or an organic acid. All of these salts may be prepared by conventional means from the corresponding compound of embodiments herein by treating, for example, the compound with the appropriate acid or base.

Pharmaceutically acceptable acids include both inorganic acids, for example hydrochloric, hydrobromic, hydroiodic, nitric, carbonic, sulfuric, phosphoric and diphosphoric acid; and organic acids, for example formic, acetic, trifluoroacetic, propionic, succinic, glycolic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, galacturonic, citric, fumaric, gluconic, glutamic, lactic, maleic, malic, mandelic, mucic, ascorbic, oxalic, pantothenic, succinic, tartaric, benzoic, acetic, xinafoic (1-hydroxy-2-naphthoic acid), napadisilic (1,5-naphthalenedisulfonic acid) and the like.

Other preferred salts according to embodiments herein are quaternary ammonium compounds wherein an equivalent of an anion (X—) is associated with the positive charge of the N atom. X— may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X— is preferably an anion selected from chloride, bromide, iodide, sulphate, nitrate, acetate, maleate, oxalate, succinate or trifluoroacetate. More preferably X— is chloride, bromide, trifluoroacetate or methanesulphonate.

The compounds of embodiments herein may exist in both unsolvated and solvated forms. The term solvate is used herein to describe a molecular complex comprising a compound of embodiments herein and an amount of one or more pharmaceutically acceptable solvent molecules. The term hydrate is employed when said solvent is water. Examples of solvate forms include, but are not limited to, compounds of embodiments herein in association with water, acetone, dichloromethane, 2-propanol, ethanol, methanol, dimethylsulfoxide (DMSO), ethyl acetate, acetic acid, ethanolamine, or mixtures thereof. It is specifically contemplated that in embodiments herein one solvent molecule can be associated with one molecule of the compounds of embodiments herein, such as a hydrate.

In some embodiments herein one solvent molecule can be associated with one molecule of the compound described herein, such as a hydrate. In some embodiments, more than one solvent molecule may be associated with one molecule of the compound described herein, such as a dihydrate. Additionally, in some embodiments herein less than one solvent molecule may be associated with one molecule of the compound described herein, such as a hemihydrate. Furthermore, solvates of embodiments herein are contemplated as solvates of the compound described herein that retain the biological effectiveness of the non-solvate form of the compounds.

The term “subject” as used herein includes, but is not limited to, humans and non-human vertebrates such as wild, domestic, and farm animals. In certain embodiments, the subject described herein is an animal. In certain embodiments, the subject is a mammal. In certain embodiments, the subject is a human. In certain embodiments, the subject is a non-human animal. In certain embodiments, the subject is a non-human mammal. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In another embodiment, the subject is a research animal such as a rodent, dog, or non-human primate. In certain embodiments, the subject is a non-human transgenic animal such as a transgenic mouse or transgenic pig.

The phrase “therapeutically effective” or “therapeutically effective amount” or “effective amount” of a composition is a predetermined amount calculated to achieve the desired effect (e.g., reduce discomfort and/or appearance of abnormalities of the skin). The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. A therapeutically effective amount of compound of this invention is typically an amount such that when it is administered in a physiologically tolerable excipient composition, it is sufficient to achieve an effective local concentration in the tissue.

Unless otherwise indicated, the term “skin” means that outer integument or covering of the body, consisting of the stratum corneum, epidermis and dermis, and resting upon subcutaneous tissue.

The terms “treat,” “treated,” or “treating” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects.

Embodiments herein are directed to compounds and pharmaceutical compositions that inhibit JAK1 and/or JAK3 activity, together with methods of using the compounds. Some embodiments include methods for the treatment of diseases in a patient by topically administering the JAK1 and/or JAK3 inhibitor compound described herein.

### Methods of Treatment

In one aspect, the present disclosure provides a method of treating a skin condition caused by an immune deficiency in a human subject. The method involves topically administering to a region of the subject affected by the skin condition a composition comprising about 0.5% to about 4% of a ethyl (R)-4-((1-(2-cyanoacetyl)piperidin-3-yl)amino)-1H-pyrrolo[2,3-b]pyridine-5-carboxylate having a structure of Compound I (hereafter “Compound I”) or a pharmaceutically acceptable derivative thereof.

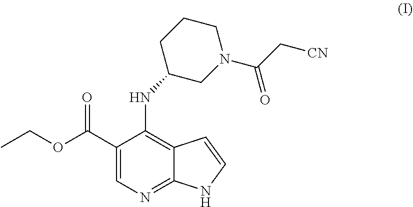 [SMILES: CCOC(=O)c1cnc2[nH]ccc2c1N[C@@H]1CCCN(C(=O)CC#N)C1]

Topical administration of a Compound I composition is therapeutically effective to achieve a desired effect. A therapeutically desired effect may include, without limitation, an improvement in one or more of the size, overall body area affected, severity, redness, flakiness, scaliness, discomfort, itchiness, or soreness of a skin lesion associated with the skin condition.

Another aspect of the present disclosure provides a method of reducing the severity and extent of skin lesions caused by an immune deficiency in a human subject. The method involves topically administering to a human subject having the immune deficiency a composition comprising about 0.5% to about 4% of Compound I or a pharmaceutically acceptable derivative, salt, or solvate thereof. Topical administration of the Compound I composition is therapeutically effective to achieve a desired effect, e.g., prevent or reduce skin lesion formation, prevent or reduce the severity of the skin lesion formation.

In accordance with these aspects of the disclosure, the skin condition treated in accordance with the methods and formulations disclosed herein is selected from atopic dermatitis, vitiligo, or alopecia areata.

Atopic dermatitis (AD) is a common, chronic skin disorder caused by complex genetic, immunological, and environmental interactions. AD is a chronic, relapsing pruritic inflammatory skin disease involving inflammation and skin barrier defects in relation to environmental stimuli. In AD, the outer layer of skin (the corneal layer), which normally acts to keep foreign substances such as bacteria, viruses, and allergens from getting into the body, is weak and more susceptible to inflammation caused by immune cells in the body reacting to these foreign substances. AD can be characterized as mild, moderate, or severe, based on skin coverage and severity of symptoms. Mild AD affects a small area of skin and may be itchy or red once in a while. However, moderate and severe AD cover larger areas of skin and are itchy more often, with periods of intense itching.

Lesional skin from patients with AD contains elevated levels of pro-inflammatory cytokines and cellular infiltrates of CD4+T cells that propagate disease pathophysiology. The cytokine microenvironment in AD appears complex, with patients presenting with different cytokine signatures at different stages of the disease.

As described herein, the Janus kinase (JAK) family of signal transducers presents is an advantageous treatment target since a variety of cytokines exert their biological effects through the JAK—signal transducer and activator of transcription (STAT) pathway. The JAK family comprises 4 cytoplasmic tyrosine kinases: JAK1, JAK2, JAK3, and TYK2; there are 7 STAT proteins: STAT 1, 2, 3, 4, 5a, 5b, and 6. The JAK-STAT signaling molecules interact downstream of numerous cytokines involved in the autoimmune and inflammatory responses. AD is driven particularly by cytokines such as IL-4, IL-13, IL-15, and interferon gamma. All of these cytokines are largely regulated by JAK1 and JAK3. As such, administration of the JAK1/3 inhibitor described herein, i.e., Compound I, is particular valuable in controlling the associated inflammation found in AD and in restoring skin barrier function. The JAK1/3 inhibitor disclosed here also has utility in treating other chronic skin conditions such as alopecia, wherein the immune system attacks hair follicles resulting in hair loss, and vitiligo, involving the loss of melanocytes and skin pigmentation.

The following disclosure and description is provided based at least in part on the clinical observation that topical administration of a therapeutically effective amount of a composition comprising Compound I, a potent inhibitor of janus kinase 1 (JAK1) and janus kinase 3 (JAK3) to the skin is effective in treating atopic dermatitis, vitiligo, and alopecia areata. Compound I competes with adenosine triphosphate at its binding site within the kinase domain, therefore, inhibiting phosphorylation of and the activation of JAKs, recruitment of STATs, and downstream effects on gene expression. In parallel, various formulations have been developed that ensure delivery of Compound I to the right target site and that dosage integrity, drug transport, and active duration are maintained and sufficient to improve patient symptoms. Additional studies were undertaken to ensure physical and chemical stability as well as assess the aesthetic appeal to potential end users.

Compound I is disclosed in U.S. Patent Application Publication No. 20190135808A1 (Example 117), which is hereby incorporated by reference in its entirety. Compound I, has a chemical formula of C₁₈H₂₁N₅O₃ and a molecular weight of 355.40 g/mol.

As used herein, “Compound I” refers to ethyl (R)-4-((1-(2-cyanoacetyl)piperidin-3-yl)amino)-1H-pyrrolo[2,3-b]pyridine-5-carboxylate and includes both the free base form and any pharmaceutically acceptable salt of ethyl (R)-4-((1-(2-cyanoacetyl)piperidin-3-yl)amino)-1H-pyrrolo[2,3-b]pyridine-5-carboxylate. For example, Compound I may be administered to a subject as an acid addition salt of hydrochloric acid, hydrobromic acid, sulfuric acid, nitric acid, carbonic acid, phosphoric acid, and the like, or an organic acid such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, or salicylic acid. As used herein, “Compound I” includes crystalline, semi-crystalline, or amorphous Compound I. Crystalline or semi-crystalline Compound I may be in the form of any pharmaceutically acceptable polymorph of Compound I or mixtures of polymorphs of Compound I.

A therapeutically effective amount or therapeutic application of a Compound I composition as described encompasses the amount of Compound I effective to reduce the severity and extent of AD related symptoms according to the EASI (Eczema Area and Severity Index) or a modified version thereof (e.g., excluding evaluation of particular body areas such as head (neck, face, scalp), palms of hands, soles of feet, groin, and genitalia). Modified versions of the EASI may include evaluation, for example, of trunk, upper extremities (arms), and lower extremities (legs). Extent of symptoms may be measured by providing each body region evaluated with a score of 0 to 6, based on the percentage involvement of that region. For example, the following Area Scores may be given: (0): 0%; (1): 1%-9%; (2): 10%-29%; (3): 30%-49%; (4): 50%-69%; (5): 70%-89%; (6): 90%-100%. The severity may be measured on a scale of 0 (least) to 3 (most) by providing the severity of each of four particular symptoms: erythema (S_{erythema}), edema/papulation (S_{edema/papulation}), excoriation (S_{excoriation}), and lichenification (S_{lichenification}).

EASI score may be calculated by the following formulas, for example, when evaluating the trunk, upper extremities, and lower extremities:

Sₜᵣᵤₙₖ=(S_{erythema}+S_{edema/papulation}+S_{excoriation}+S_{lichenification})×Area Score×0.3

S_{upper extremities}=(S_{erythema}+S_{edema/papulation}+S_{excoriation}+S_{lichenification})×Area Score×0.2

S_{lower extremities}=(S_{erythema}+S_{edema/papulation}+S_{excoriation}+S_{lichenification})×Area Score×0.4

EASI score=Sₜᵣᵤₙₖ+S_{upper extremities}+S_{lower extremities}

Thus, in accordance with the methods disclosed herein, topical administration of a composition comprising about 0.5% to about 4% Compound I to a region of a subject affected by a skin condition, such as AD, results in an improvement of at least 25%, at least 30%, at least 35%, at least 40%, at least 45%, at least 50%, at least 55%, at least 60%, at least 65%, at least 70%, at least 75%, or greater than 75% in the subject's EASI score from the baseline score, i.e., the score prior to commencing treatment.

In another embodiment, a therapeutically effective amount of Compound I is the amount effective to reduce the discomfort of the skin condition caused by itchiness according to a self-reporting PP-NRS (Peak Pruritus Numerical Rating Scale). The PP-NRS is designed to measure peak pruritus or worst itch over a 24-hour period based on a scale of 0 (no itch) to 10 (worst itch). In some embodiments, topical administration of a composition comprising about 0.5% to about 4% Compound I to a region of a subject affected by a skin condition, such as AD, results in a reduction of at least one point, at least two points, at least three points, or a reduction of greater than three points on the PP-NRS scale.

In another embodiment, a therapeutically effective amount of Compound I is the amount effective to reduce the overall amount of body surface area (BSA) affected by the skin condition. The BSA affected by the skin condition, for example, AD, may be estimated by a clinician or clinical investigator, doctor, or other medical professional using the handprint method, which estimates that the area of a patient's full handprint (fingers and thumbs together) constitutes 1% of their total BSA. In some embodiments, topical administration of a composition comprising about 0.5% to about 4% Compound I to a region of a subject affected by a skin condition, such as AD, results in a significant reduction of their total BSA affected by the condition.

In another embodiment, a therapeutically effective amount of Compound I is the amount that improves the overall appearance of lesions as assessed by an independent person (e.g., Investigator's Global Assessment or IGA), such as a clinician or clinical investigator, doctor, or other medical professional. IGA scoring may be based, for example, on the following criteria to provide a numerical representation of appearance.

0—clear: no inflammatory signs of atopic dermatitis (no erythema, no induration/papulation, no lichenification, no oozing/crusting). Post-inflammatory hyperpigmentation and/or hypopigmentation may be present.

1—almost clear: barely perceptible erythema, barely perceptible induration/papulation, and/or minimal lichenification. No oozing or crusting.

2—mild: slight but definite erythema (pink), slight but definite induration/papulation, and/or slight but definite lichenification. No oozing or crusting;

3—moderate: clearly perceptible erythema (dull red), clearly perceptible induration/papulation, and/or clearly perceptible lichenification. Oozing and crusting may be present.

4—Severe: marked erythema (deep or bright red), marked induration/papulation, and/or marked lichenification. Disease is widespread in extent. Oozing or crusting may be present.

In some embodiments, topical administration of a composition comprising about 0.5% to about 4% Compound I to a region of a subject affected by a skin condition, such as AD, results in a reduction in the IGA score of the subject. In some embodiments, treatment with the Compound I composition as described herein results in at least a reduction in the IGA score of the subject of at least one point.

Additionally, or alternatively, a therapeutically effective amount of Compound I is an amount sufficient to reduce the frequency or severity of lesion outbreaks, for example, smaller lesions, fewer lesions, or less itchy or noticeable lesions.

A therapeutically effective amount of Compound I is an amount such that when it is administered in a topical formulation, it is sufficient to achieve an effective local concentration in the skin tissue. In any embodiment, a therapeutically effective amount of Compound I reduces the levels of pro-inflammatory cytokines and cellular infiltrates of CD4+ T cells at or near the site of administration. In any embodiment, a therapeutically effective amount of Compound I reduces the levels of one or more of IL-4, IL-13, IL-15, and interferon gamma at or near the site of administration. In any embodiment, a therapeutically effective amount of Compound I increases filaggrin expression at or near the site of administration.

Particular dosing amounts and regimens may vary from subject to subject depending on variables such as, but not limited to, severity of lesions, sensitivities to inactive ingredients, sensitivity to Compound I, frequency of lesion outbreaks, frequency and/or severity of adverse events, weight, age, gender, co-morbidity with other diseases or conditions, use of adjuvant therapies to treat AD, use of other non-AD related medications, and the like.

For example, Compound I may be administered to a subject in need thereof at a dose of about 20 mg/day to about 320 mg/day, which includes about 40 mg/day to about 280 mg/day, about 80 mg/day to about 240 mg/day, about 120 mg/day to about 200 mg/day, about 40 mg/day to about 160 mg/day, about 80 mg/day to about 160 mg/day, or about 160 mg/day to about 320 mg/day. Administration of Compound I may be performed once a day, twice a day, three times a day, or more often. In any embodiment, the dose per day, as listed above, may be divided times the number of administrations in a day to yield a dose per administration. For example, a dose of about 40 mg/day to about 320 mg/day equates to a dose of about 20 mg/administration to about 160 mg/administration when applied twice daily.

Alternatively, dosing instructions may be provided on a per-administration basis. Therefore, in yet another aspect, the present disclosure provides a method of treating atopic dermatitis, vitiligo, or alopecia areata in a subject in need thereof comprising topically administering to the subject about 10 mg per administration to about 160 mg per administration of Compound I or a pharmaceutically acceptable derivative, salt, or solvate thereof. Administration of Compound I by way of topical administration may be performed once a day, twice a day, three times a day, or more often. Each interval between administrations may be the same as other intervals or may be different. For example, when administered twice per day, a second administration may occur about 12 hours after the first administration. Alternatively, the administrations may be carried out at equal intervals during waking hours only, such as from 6:00 am to about 10:00 pm. Suitable doses include about 10 mg per administration to about 180 mg per administration, which includes about 20 mg/administration, about 60 mg/administration, about 80 mg/administration, about 100 mg/administration, about 120 mg/administration, and about 140 mg/administration. One of skill in the art will be able to optimize the dosing amount as well as the dosing interval to achieve favorable treatment for the subject to which the Compound I is being administered.

Compound I may be administered to a subject in the form of a topical formulation, such as, but not limited to, a topical solution, an ointment, a cream, a gel, a gel-cream, a foam, or the like. For example, the topical formulation may be a topical solution. The topical formulation may be applied directly to the skin, for example, to a lesion of the skin or a location where a lesion commonly appears. The topical formulation may be applied to the skin, for example, through use of a dropper. Optionally, and if desired, a pumping mechanism may additionally be supplied with the plastic bottle/cap such that the cap may be removed and replaced securely with the pumping mechanism for delivering the topical formulation as a spray. Upon administration, the topical formulation may be rubbed into the skin to facilitate absorbance of Compound I into the affected area and skin layers. The volume of the topical formulation that is administered to the skin topically may be calculated based on the desired dose of Compound I and the concentration of Compound I in the formulation. In general, about 0.25 mL per administration to about 8 mL per administration of a topical formulation comprising Compound I may be topically administered to a subject.

Compound I may be present in the topical formulation at a concentration of about 0.01% w/w to a maximum concentration representing maximum equilibrium saturation solubility at room temperature in the vehicle or vehicles selected. One of ordinary skill in the art will be able to readily determine maximum equilibrium saturation solubility of Compound I in any chosen solvent or mixture of solvents. In particularly useful embodiments, Compound I may be present at or less than the concentration representing about 85% of its maximum equilibrium saturation solubility. In particular, Compound I may be present in the topical formulation at a concentration of about 0.01% w/w to about 10% w/w, for example, about 1% w/w, about 2% w/w, about 3% w/w, about 4% w/w, about 5% w/w, about 6% w/w, about 7% w/w, about 8% w/w, about 9% w/w or about 10% w/w. Compound I may be present in the topical formulation at a concentration of about 0.01% w/w to about 4% w/w, such as about 0.5% w/w to about 4% w/w, about 0.5% w/w to about 3.5% w/w, about 0.5% w/w to about 2% w/w, about 1.5% w/w to about 2.5% w/w, or about 1% w/w to about 2% w/w. In any embodiment, Compound I may be present in the topical formulation at a concentration of about 0.5% w/w, 0.6% w/w, 0.7% w/w, 0.8% w/w, 0.9% w/w, 1.0% w/w, 1.1% w/w, 1.2% w/w, 1.3% w/w, 1.4% w/w, 1.5% w/w, 1.6% w/w, 1.7% w/w, 1.8% w/w, 1.9% w/w, 2.0% w/w, 2.1% w/w, 2.2% w/w, 2.3% w/w, 2.4% w/w, 2.5% w/w, 2.6% w/w, 2.7% w/w, 2.8% w/w, 2.9% w/w, 3.0% w/w, 3.1% w/w, 3.2% w/w, 3.3% w/w, 3.4% w/w, 3.5% w/w, 3.6% w/w, 3.7% w/w, 3.8% w/w, 3.9% w/w, 4.0% w/w, 4.1% w/w, 4.2% w/w, 4.3% w/w, 4.4% w/w, 4.5% w/w, 4.6% w/w, 4.7% w/w, 4.8% w/w, 4.9% w/w, or 5.0% w/w,. As used herein, and unless otherwise specified, % w/w refers to percent by weight of the named component when compared to the total weight of the formulation of which it is part, where the total weight of the formulation is set at 100% w/w. Compound I may be present in the topical formulation in any physical form, including but not limited to, a solvated form, a non-solvated form, or a complexed form in the topical formulation as a free base or as a salt.

In one aspect, treatment of one or more of atopic dermatitis, vitiligo, and alopecia areata in a subject in need thereof with topical Compound I may be performed on a chronic basis, for example, for ongoing treatment and prevention of symptoms of the skin condition, such as preventing the formation of lesions or reducing the severity of lesions that form when compared to untreated lesions. In another aspect, administration may be performed as needed as symptoms arise or are predicted to arise. In any embodiment, Compound I may be administered on a chronic basis with additional administrations and/or increased dosing upon occurrence of symptoms. Examples of symptoms include, but are not limited to, dry and/or scaly skin, itching, reddening of the skin, skin infections, and bleeding or oozing skin. In any embodiment, dosing may be increased or decreased based on severity of symptoms.

One exemplary dosing regimen for the treatment of atopic dermatitis includes topical administration of about 1 mL to about 8 mL of a 2% Compound I formulation twice daily to an area of the subject affected by atopic dermatitis, which includes application of a) about 1 mL of a 2% w/w Compound I formulation twice daily; b) about 2 mL of a 2% w/w Compound I formulation twice daily; c) about 3 mL of a 2% w/w Compound I formulation twice daily; d) about 4 mL of a 2% w/w Compound I formulation twice daily; e) about 5 mL of a 2% w/w Compound I formulation twice daily; f) about 6 mL of a 2% w/w Compound I formulation twice daily; g) about 7 mL of a 2% w/w Compound I formulation twice daily; and h) about 8 mL of a 2% w/w Compound I formulation twice daily.

Another exemplary dosing regimen includes topical administration of about 1 mL to about 6 mL of 2% w/w Compound I formulation three times daily to an area of the subject affected by atopic dermatitis, which includes application of a) about 1 mL of a 2% w/w Compound I formulation three times daily; b) about 2 mL of a 2% w/w Compound I formulation three times daily; c) about 3 mL of a 2% w/w Compound I formulation three times daily; d) about 4 mL of a 2% w/w Compound I formulation three times daily; e) about 5 mL of a 2% w/w Compound I formulation three times daily; and d) about 6 mL of a 2% w/w Compound I formulation three times daily.

Another exemplary dosing regimen includes administration of a lower amount of Compound I more often, for example, topical administration of about 1 mL to about 6 mL of 0.5% w/w Compound I formulation three times daily to an area of the subject affected by atopic dermatitis, which includes application of a) about 1 mL of a 0.5% w/w Compound I formulation three times daily; b) about 2 mL of a 0.5% w/w Compound I formulation three times daily; c) about 3 mL of a 0.5% w/w Compound I formulation three times daily; d) about 4 mL of a 0.5% w/w Compound I formulation three times daily; e) about 5 mL of a 0.5% w/w Compound I formulation three times daily; f) about 6 mL of a 0.5% w/w Compound I formulation three times daily; g) about 7 mL of a 2% w/w Compound I formulation three times daily; and h) about 8 mL of a 2% w/w Compound I formulation three times daily.

Yet another exemplary dosing regimen includes topical administration of about 1 mL to about 8 mL of a 0.5% w/w Compound I formulation four times daily to an area of the subject affected by atopic dermatitis, which includes application of a) about 1 mL of a 0.5% w/w Compound I formulation twice daily; b) about 2 mL of a 0.5% w/w Compound I formulation twice daily; c) about 3 mL of a 0.5% w/w Compound I formulation twice daily; d) about 4 mL of a 0.5% w/w Compound I formulation twice daily; e) about 5 mL of a 0.5% w/w Compound I formulation twice daily; f) about 6 mL of a 0.5% w/w Compound I formulation twice daily; g) about 7 mL of a 0.5% w/w Compound I formulation twice daily; and about 8 mL of a 0.5% w/w Compound I formulation twice daily.

In any embodiment, Compound I may be used as a co-therapy or adjuvant therapy to another AD-related therapy. For example, a method of treating atopic dermatitis, vitiligo, or alopecia areata in a subject in need thereof comprising topically administering to the subject a therapeutically effective amount of Compound I or a pharmaceutically acceptable derivative, salt, or solvate thereof in combination with one or more of an immunosuppressant, an antihistamine, a corticosteroid, a calcineurin inhibitor, a phosphodiesterase-4 inhibitor, dupilumab, an antimicrobial, and a humanized monoclonal antibody that blocks signaling of interleukin (IL)-4/IL-13.

### Pharmaceutical Formulations

Another aspect of the present disclosure is directed to a topical formulation comprising ethyl (R)-4-((1-(2-cyanoacetyl)piperidin-3-yl)amino)-1H-pyrrolo[2,3-b]pyridine-5-carboxylate (Compound I) or a pharmaceutically acceptable derivative, salt, or solvate thereof, and a vehicle which may comprise a primary solvent and one or more co-solvents. The formulation may further comprise one or more antioxidants, one or more antimicrobial agents, or any other desired excipients such as, but not limited to, diluents, surfactants, emulsifiers, emollients, stabilizers, viscosity modifiers, coloring agents, fragrances, or any combination thereof. In particularly useful embodiments, the topical formulation comprises an emollient.

Compound I may be present in the topical formulation at a concentration of about 0.01% w/w to a maximum concentration representing maximum equilibrium saturation solubility at room temperature in the vehicle or vehicles selected. One of ordinary skill in the art will be able to readily determine maximum equilibrium saturation solubility of Compound I in any chosen solvent or mixture of solvents. In particularly useful embodiments, Compound I may be present at or less than the concentration representing about 85% of its maximum equilibrium saturation solubility. In particular, Compound I may be present in the topical formulation at a concentration of about 0.01% w/w to about 10% w/w, for example, about 1% w/w, about 2% w/w, about 3% w/w, about 4% w/w, about 5% w/w, about 6% w/w, about 7% w/w, about 8% w/w, about 9% w/w or about 10% w/w. Compound I may be present in the topical formulation at a concentration of about 0.01% w/w to about 4% w/w, such as about 0.5% w/w to about 4% w/w, about 0.5% w/w to about 3.5% w/w, about 0.5% w/w to about 2% w/w, about 1.5% w/w to about 2.5% w/w, or about 1% w/w to about 2% w/w. In any embodiment, Compound I may be present in the topical formulation at a concentration of about 0.5% w/w, 0.6% w/w, 0.7% w/w, 0.8% w/w, 0.9% w/w, 1.0% w/w, 1.1% w/w, 1.2% w/w, 1.3% w/w, 1.4% w/w, 1.5% w/w, 1.6% w/w, 1.7% w/w, 1.8% w/w, 1.9% w/w, 2.0% w/w, 2.1% w/w, 2.2% w/w, 2.3% w/w, 2.4% w/w, 2.5% w/w, 2.6% w/w, 2.7% w/w, 2.8% w/w, 2.9% w/w, 3.0% w/w, 3.1% w/w, 3.2% w/w, 3.3% w/w, 3.4% w/w, 3.5% w/w, 3.6% w/w, 3.7% w/w, 3.8% w/w, 3.9% w/w, 4.0% w/w, 4.1% w/w, 4.2% w/w, 4.3% w/w, 4.4% w/w, 4.5% w/w, 4.6% w/w, 4.7% w/w, 4.8% w/w, 4.9% w/w, or 5.0% w/w. As used herein, and unless otherwise specified, % w/w refers to percent by weight of the named component when compared to the total weight of the formulation of which it is part, where the total weight of the formulation is set at 100% w/w. Compound I may be present in the topical formulation in any physical form, including but not limited to, a solvated form, a non-solvated form, or a complexed form in the topical formulation as a free base or as a salt.

As is understood by those skilled in the art, some excipients may serve multiple functions. For example, PEG-400 may function as a solvent and an emollient. The combination of formulation ingredients disclosed herein are selected to maximize solubility, moisturing/emollient effect while balancing the needs of sprayability and hence spreadability.

The topical formulation, as described herein, may comprise a primary solvent. Examples of suitable primary solvents include, but are not limited to, one or more polyethylene glycols (e.g., PEG-400, PEG-300), diethylene glycol monoethyl ether (e.g., TRANSCUTOL™ P or HP), or any combination thereof.

In particularly useful embodiments, the primary solvent comprises polyethylene glycol. As used herein, the term “polyethylene glycol” refers to a polymer containing ethylene glycol monomer units of formula —O—CH₂-CH₂—. Suitable polyethylene glycols may have a free hydroxyl group at each end of the polymer molecule, or may have one or more hydroxyl groups etherified with a lower alkyl (e.g., a methyl group). Suitable polyethylene glycols include, but are not limited to polyethylene glycol-200 (PEG-200), polyethylene glycol-300 (PEG-300), and polyethylene glycol-400 (PEG-400). In one embodiment, the polyethylene glycol solvent is primarily PEG-400 or comprises a mixture of PEG-400 and diethylene glycol monoethyl ether. The number following the dash in the name refers to the average molecular weight of the polymer (i.e., PEG-400 has an average molecular weight of about 400 g/mol). In some embodiments, SUPER REFINED™ grades of polyethylene glycol from Croda (Edison, NJ, USA) are used.

The primary solvent may constitute at least about 50 w/w % of the topical formulation. For example, the primary solvent of the topical formulation may comprise about 50 w/w % to about 90 w/w %, about 65 w/w % to about 90 w/w %, about 60 w/w % to about 80 w/w %, or about 65 w/w % to about 70 w/w %, including any concentrations or concentration ranges there within. When the primary solvent comprises a mixture of polyethylene glycol and diethylene glycol monoethyl ether, the primary solvent may comprise about 40% by weight to about 50% by weight of polyethylene glycol (e.g., about 40 w/w %, about 42 w/w %, about 44 w/w %, about 46 w/w % about 48 w/w %, or about 50 w/w %, as well as any range there between such as from about 40 w/w % to about 42 w/w %, from about 42 w/w % to about 44 w/w %, from about 44 w/w % to about 46 w/w %, from about 46 w/w % to about 48 w/w %, and from about 48 w/w % to about 50 w/w %). Additionally, or alternatively, the ratio of polyethylene glycol-to-diethylene glycol monoethyl ether may be about 0.7:1 to about 1:1.

One or more co-solvents may also be included in the topical formulation. Examples of suitable co-solvents include, but are not limited to, dimethyl isosorbide, benzyl alcohol, ethanol, phenoxyethanol, propylene glycol, propylene carbonate, dimethyl sulfoxide, water, and any mixture thereof. In particularly useful embodiments, a topical formulation may comprise about 10 w/w % to about 20 w/w % (e.g., about 10 w/w %, about 11 w/w %, about 12 w/w %, about 13 w/w %, about 14 w/w %, about 15 w/w %, about 16 w/w %, about 17 w/w %, about 18 w/w %, about 19 w/w %, or about 20 w/w %) dimethyl isosorbide; about 3 w/w % to about 6 w/w % (e.g., about 3 w/w %, about 4 w/w %, about 5 w/w %, or about 6 w/w %) propylene glycol; and about 1 w/w % to about 3 w/w % (e.g., 1 w/w %, about 2 w/w %, or about 3 w/w %) benzyl alcohol. Collectively, the one or more co-solvents may comprise about 20 w/w % to 30 w/w % of the topical formulation.

A suitable topical formulation may comprise one or more emollients. In one embodiment, the emollient is glycerol. Other suitable emollients include, without limitation, glyceryl monostearate, isopropyl myristate, isopropyl palmitate, isopropyl isostearate, diisopropyl adipate, diisopropyl dimerate, maleated soybean oil, octyl palmitate, cetyl lactate, cetyl ricinoleate, tocopheryl acetate, cetyl acetate, tocopheryl linoleate, wheat germ glycerides, arachidyl propionate, myristyl lactate, decyl oleate, propylene glycol, propylene glycol ricinoleate, isopropyl lanolate, pentaerythrityl tetrastearate, neopentylglycol dicaprylate/dicaprate, caprylyl glycol, isononyl isononanoate, isotridecyl isononanoate, myristyl myristate, octyl dodecanol, sucrose esters of fatty acids and octyl hydroxystearate. The one or more emollients may be included in the topical formulation at about 6 w/w % to about 10 w/w % (e.g., about 6 w/w %, about 7 w/w %, about 8 w/w %, about 9 w/w %, or about 10 w/w %.

An antioxidant may be included in the topical formulation at a concentration of about 0.01 w/w % to about 1 w/w %, such as from about 0.1 w/w % to about 0.5 w/w %. Examples of suitable antioxidants include, but are not limited to butylated hydroxytoluene (BHT), butylated hydroxyanisole (BHA), vitamin C and its derivatives, vitamin E and its derivatives, and propyl gallate. In particularly useful embodiments, the antioxidant comprises butylated hydroxytoluene.

Optionally, an antimicrobial preservative may be present. Examples of suitable antimicrobial preservatives include benzyl alcohol (which also may be considered a solvent) benzalkonium chloride, benzoic acid, centrimide, chlorocresol, chlorobutanol, glycerin, propylene glycol, phenoxyethanol, methylparaben, ethylparaben, butylparaben, propylparaben, and a combination thereof.

The topical formulations as described herein preferably have an apparent viscosity of about 100 mPa·s or less, more preferably about 80 mPa·s or less. For example, a topical formulation as described herein may have an apparent viscosity of about 1 mPa·s to about 100 mPa·s , about 1 mPa·s to about 80 mPa·s, about 5 mPa·s to about 60 mPa·s, about 10 mPa·s to about 80 mPa·s, about 10 mPa·s to about 40 mPa·s, or about 15 mPa·s to about 50 mPa·s.

Various topical therapeutically effective formulations are contemplated as within the scope of this invention. In one embodiment, a suitable topical formulation comprises a therapeutically effective amount of Compound I, a salt thereof, or a solvate thereof; a primary solvent comprising one or both of a mixture of polyethylene glycols having an average molecular weight of about 200 g/mol to about 400 g/mol and diethylene glycol monoethyl ether; a co-solvent comprising one or more of benzyl alcohol, ethanol, and phenoxyethanol; dimethyl isosorbide; propylene glycol; glycerol; and an antioxidant. Such a formulation may have an apparent viscosity of about 1 mPa·s to about 100 mPa·s.

In any embodiment, a suitable topical formulation of the present disclosure comprises about 0.5 w/w % to about 3.5 w/w % Compound I or a pharmaceutically acceptable derivative thereof; about 68 w/w % to about 70 w/w % of a primary solvent comprising one or both of a mixture of polyethylene glycols having an average molecular weight of about 200 g/mol to about 400 g/mol and diethylene glycol monoethyl ether; about 2 w/w % of benzyl alcohol; about 15 w/w % of dimethyl isosorbide; about 5 w/w % of propylene glycol; about 8 w/w % to about 10 w/w % of glycerol; and about 0.05 w/w % to about 0.5 w/w % of an antioxidant. Such a formulation may have an apparent viscosity of about 10 mPa·s to about 100 mPa·s.

In any embodiment, a suitable topical formulation of the present disclosure comprises about 0.5 w/w % to about 3.5 w/w % Compound I or a pharmaceutically acceptable derivative thereof; a mixture of about 28 w/w % to about 34 w/w % of polyethylene glycols having an average molecular weight of about 300 g/mol to about 400 g/mol; about 35 w/w % to about 40 w/w % diethylene glycol monoethyl ether; about 2 w/w % of benzyl alcohol; about 15 w/w % of dimethyl isosorbide; about 5 w/w % of propylene glycol; about 8 w/w % to about 10 w/w % of glycerol; and about 0.05 w/w % to about 0.5 w/w % of an antioxidant, such as butylated hydroxytoluene. Such a formulation may have an apparent viscosity of about 1 mPa·s to about 40 mPa·s.

In any embodiment, a suitable topical formulation of the present disclosure comprises about 0.5 w/w % to about 3.5 w/w % Compound I or a pharmaceutically acceptable derivative thereof; about 68 w/w % to about 70 w/w % of a primary solvent consisting of polyethylene glycols having an average molecular weight of about 300 g/mol to about 400 g/mol; about 2 w/w % of benzyl alcohol; about 15 w/w % of dimethyl isosorbide; about 5 w/w % of propylene glycol; about 8 w/w % to about 10 w/w % of glycerol; and about 0.05 w/w % to about 0.5 w/w % of an antioxidant, such as butylated hydroxytoluene. Such a formulation may have an apparent viscosity of about 1 mPa·s to about 100 mPa·s.

Additional exemplary topical formulations of the present disclosure are provided in Table 1 of the Examples herein.

The topical formulation may be supplied in a plastic (e.g., HDPE) or glass bottle closed with a plastic (e.g., polypropylene, HDPE) cap. The topical formulation may be applied to the skin, for example, through use of a dropper. Optionally, and if desired, a pumping mechanism may additionally be supplied with the plastic bottle/cap such that the cap may be removed and replaced securely with the pumping mechanism for delivering the topical formulation as a spray. As such, a sprayable formulation should exhibit a compatible viscosity, as disclosed above, with the pumping/spraying mechanism utilized. One of skill in the art will be familiar with a variety of commercially available pump/spray actuators that may be used for topical delivery of the formulations disclosed herein. Application of the topical formulation may be controlled via a valve providing continuous or metered spray.

The topical formulations disclosed herein have demonstrated prolonged chemical and physical stability under various storage conditions. For example, various topical formulations disclosed herein were found to be stable for up to 6 months when stored at 25° C. at 60% relative humidity (RH) and at 40° C. at 75% RH when measured by HPLC. In particular, the recovery of Compound I in one or more of the formulations as described herein may measure to be about 95% to about 100% after storage for 6 months at both 25° C./60% RH and 40° C./75% RH. Macroscopic and microscopic appearance after storage under these conditions may remain constant.

Methods for preparing the topical formulations disclosed herein may be any suitable and known to one of ordinary skill in the art. For example, a topical formulation as disclosed herein may be prepared by weighing the alcohol and glycol-containing solvents/co-solvents (e.g., benzyl alcohol, ethanol, phenoxyethanol, polyethylene glycol, diethylene glycol monoethyl ether) along with additional optional antioxidant (e.g., BHT) and/or antibacterial agent. These components may be mixed generally in any order to form a substantially homogenous mixture. If needed, the mixture may be heated to facilitate dissolution.

The topical formulations disclosed herein may be useful in treating conditions involving janus kinase signaling, in particular, signaling pathways of janus kinase 1 (JAK1) and janus kinase 3 (JAK3). Examples of such conditions include, but are not limited to, atopic dermatitis, vitiligo, and alopecia areata. Suitable formulations may comprise a therapeutically effective amount of Compound I.

Advantageously, the topical formulations have been developed to meet a complex set of criteria. First, the topical formulations described herein exhibit excellent physical and chemical stability, both of the formulation and of Compound I in the formulation to maintain efficacy throughout storage. Further, the formulations have been developed with permeation agents (e.g., dimethyl isosorbide, diethylene glycol monomethyl ether, and propylene glycol) in effective amounts to effect sufficient permeation of Compound I into the skin while minimizing system introduction. Yet further, the formulations have been developed with patient compliance and comfort in mind. The inclusion of an emollient provides a formulation that is easy to use, alleviates symptoms of the underlying condition to be treated with Compound I, and therefore, a patient is more likely to use the product. Each of these aspects, when combined, provides a useful topical formulation for treatment of various skin disorders and conditions with Compound I that is not currently available.

As stated prior, the combination of formulation ingredients disclosed herein are selected to maximize solubility, moisture/emollient effect while balancing the needs of sprayability and hence spreadability. In certain embodiments, application of the formulation in the absence of Compound I results in reduction of the severity and extent of skin lesions caused by an immune deficiency in a human subject. In certain embodiments, the formulation may exclude Compound I and further comprise a vehicle which may comprise a primary solvent and one or more co-solvents. The formulation may further comprise one or more antioxidants, one or more antimicrobial agents, antifungal agents, or any other desired excipients such as, but not limited to, diluents, fillers, lubricants, surfactants, emulsifiers, emollients, stabilizers, viscosity modifiers, coloring agents, fragrances, or any combination thereof. In particularly useful embodiments, the topical formulation comprises an emollient.

In another embodiment, the formulation lacking Compound I comprises about 68 w/w % to about 70 w/w % of a primary solvent comprising one or both of a mixture of polyethylene glycols having an average molecular weight of about 200 g/mol to about 400 g/mol and diethylene glycol monoethyl ether; about 2 w/w % of benzyl alcohol; about 15 w/w % of dimethyl isosorbide; about 5 w/w % of propylene glycol; about 8 w/w % to about 10 w/w % of glycerol; and about 0.05 w/w % to about 0.5 w/w % of an antioxidant. Such a formulation may have an apparent viscosity of about 10 mPa·s to about 100 mPa·s.

In another embodiment, the formulation lacking Compound I comprises a mixture of about 28 w/w % to about 34 w/w % of polyethylene glycols having an average molecular weight of about 300 g/mol to about 400 g/mol; about 35 w/w % to about 40 w/w % diethylene glycol monoethyl ether; about 2 w/w % of benzyl alcohol; about 15 w/w % of dimethyl isosorbide; about 5 w/w % of propylene glycol; about 8 w/w % to about 10 w/w % of glycerol; and about 0.05 w/w % to about 0.5 w/w % of an antioxidant, such as butylated hydroxytoluene. Such a formulation may have an apparent viscosity of about 1 mPa·s to about 40 mPa·s.

In yet another embodiment, the formulation lacking Compound I comprises about 68 w/w % to about 70 w/w % of a primary solvent consisting of polyethylene glycols having an average molecular weight of about 300 g/mol to about 400 g/mol; about 2 w/w % of benzyl alcohol; about 15 w/w % of dimethyl isosorbide; about 5 w/w % of propylene glycol; about 8 w/w % to about 10 w/w % of glycerol; and about 0.05 w/w % to about 0.5 w/w % of an antioxidant, such as butylated hydroxytoluene. Such a formulation may have an apparent viscosity of about 1 mPa·s to about 100 mPa·s. To facilitate a better understanding of the embodiments of the present invention, the following examples of preferred or representative embodiments are given. In no way should the following examples be read to limit, or to define, the scope of the invention.

Although the present invention has been described in considerable detail with reference to certain preferred embodiments thereof, other versions are possible. Therefore, the spirit and scope of the appended claims should not be limited to the description and the preferred versions contained within this specification.

### EXAMPLES

### Formulation Characterization Methods for Examples 1-3

Surface tension was measured at 25° C. using the Kinexus Ultra+Rheometer S/N MAL1102043 by Malvern Instruments (DeNouy Ring geometry).

Viscosity was measured at 25° C.±1.0° C. on a Brookfield model DV 2T/LV viscometer with Rheocalc T.1.2.19 software.

Storage stability was measured for various topical formulations stored in amber glass bottles closed with a polypropylene cap glass vials, stored for up to 24 months at 25° C./60% RH and 40° C./75% RH for 24 and 6 months respectively. Compound I content and peak purity were measured via HPLC (method described below), micro- and macroscopic appearance were assessed, benzyl alcohol and butylated hydroxytoluene contents benzyl were measured.

Permeability Studies: Ex Vivo/In Vitro permeability studies were carried out by mounting human skin on flow through diffusion cells (MEDFLUX-HT®). Test formulations were placed on the top surface of the skin while a receptor solution (citrate/phosphate buffer pH 4.0+0.1% polyethylene glycol hexadecyl ether (BRIJ®)) was perfused underneath to capture any Compound I that permeated through the skin. The skin itself, after being removed from the flow cell, was subjected to an extraction (10:90 v/v acetonitrile: water) procedure to quantify the amount of Compound I remaining in the skin. Compound I content in receptor solutions and extraction solutions was quantified by HPLC (method described below).

HPLC Methods: An HPLC Waters Alliance equipped with a Waters™ XBRIDGE® BEH C₁₈, 35 μm, 150×4.6 mm column was utilized. Compound I was detected via UV at a wavelength of 235 nm. Column temperature was maintained at 40±5° C. Mobile phase A was 0.1% TFA in water, mobile phase B was 100% methanol and samples were analyzed via gradient elution at a flow rate of 1.0 mL/min.

### Example 1: Example Formulations

Table 1 below reports various specific examples of topical formulations, some of which represent exemplary embodiments of the topical formulations as described above, that may be administered to a subject to treat a skin condition caused by an immune deficiency disease, such as atopic dermatitis, alopecia areata, or vitiligo.

TABLE 1

| | Formulation No. | | | | | | |
|---|---|---|---|---|---|---|---|
| | 1 | 2 | 3 | 4 | 5 | 6 | 7 |
| Component | Amount (% w/w) | | | | | | |
| Compound I | 2 | 2 | 2 | 2 | 2 | 2 | 2 |
| Ethanol | 4 | — | 4 | — | — | — | — |
| Benzyl Alcohol | — | 2 | — | 2 | 2 | 2 | 2 |
| Phenoxyethanol | — | — | — | — | 1 | 1 | 1 |
| Propylene carbonate | — | — | — | — | — | — | — |
| PEG-400 | 65.5 | 61 | 65.4 | 67.4 | — | 28.5 | 33.5 |
| PEG-300 | — | — | — | — | 28.5 | — | |
| Glycerol | 8.5 | — | 8.5 | 8.5 | 8.4 | 8.4 | 8.4 |
| Dimethyl Isosorbide | 15 | — | 15 | 15 | 15 | 15 | 15 |
| Propylene Glycol | 5 | — | 5 | 5 | 5 | 5 | 5 |
| Diethylene glycol | — | 25 | — | — | 40 | 40 | 35 |
| monoethyl ether, | | | | | | | |
| (TRANSCUTOL ® P) | | | | | | | |
| BHT | — | — | 0.1 | 0.1 | 0.1 | 0.1 | — |
| Dimethyl Sulfoxide | — | — | — | — | — | — | 0.1 |
| Water (pH 4) | — | 10 | — | — | — | — | — |
| Total % | 100.0 | 100.0 | 100.0 | 100.0 | 100.0 | 100.0 | 100.0 |

Measured Properties

| | | | | | | | |
|---|---|---|---|---|---|---|---|
| Viscosity/25° C. (mPa · s) | — | — | — | 78 | — | 26.8 | 21.3 |
| Surface Tension (N/m) | — | — | — | 0.096 | — | 0.035 | — |

Formulation No.

| | | | | | |
|---|---|---|---|---|---|
| 8 | 9 | 10 | 11 | 12 | 13 |

| | |
|---|---|
| Component | Amount (% w/w) |

| | | | | | | |
|---|---|---|---|---|---|---|
| Compound I | 2 | 4.0 | 3.8 | 3.1 | 2.8 | 0.5 |
| Ethanol | — | — | — | — | — | — |
| Benzyl Alcohol | 2 | 2.5 | 2.5 | 2.5 | 2.5 | 2 |
| Phenoxyethanol | — | 1.2 | 1.3 | 1.3 | 1.3 | — |
| Propylene carbonate | — | 6.2 | 6.3 | 6.3 | 6.3 | — |
| PEG-400 | 28.5 | — | — | — | — | 28.5 |
| PEG-300 | | — | — | — | — | — |
| Glycerol | 9.4 | 6.2 | 6.3 | 6.3 | 6.3 | 9.4 |
| Dimethyl Isosorbide | 15 | 12.5 | 12.5 | 17.6 | 17.7 | 15 |
| Propylene Glycol | 5 | 12.5 | 12.5 | 12.6 | 12.6 | 5 |
| Diethylene glycol | 40 | 49.8 | 50.0 | 50.3 | 50.5 | 39.5 |
| monoethyl ether, | | | | | | |
| (TRANSCUTOL ® P) | | | | | | |
| BHT | — | 5.0 | 5.0 | — | — | — |
| Dimethyl Sulfoxide | 0.1 | — | — | — | — | 0.1 |
| Water (pH 4) | — | — | — | — | — | — |
| Total % | 100.0 | 100.0 | 100.0 | 100.0 | 100.0 | 100.0 |

Measured Properties

| | | | | | | |
|---|---|---|---|---|---|---|
| Viscosity/25° C. (mPa · s) | 18.9 | — | — | — | — | — |
| Surface Tension (N/m) | 0.098 | — | — | — | — | — |

### Example 2: Preparation of Formulation 4

23.62 kg of SUPER REFINED™ PEG-400 from Croda (Snaith, United Kingdom) and 1.75 kg of SUPER REFINED™ propylene glycol (from Croda) were combined in a mixer. 700 g benzyl alcohol and 35 g butylated hydroxytoluene (BHT) were added to the mixer. The resulting mixture was stirred until all BHT was dissolved and a clear solution was formed. The contents were mixed. Separately, 707.07 g of Compound I and 3.25 kg of SUPER REFINED™ ARLASOLVE™ dimethyl isosorbide (from Croda) were combined in a vessel and mixed until a uniform dispersion was obtained. The dispersion was added to the clear solution in the mixer using 2000 g of additional dimethyl isosorbide to rinse the vessel. The combined mixture was mixed while heating until Compound I was dissolved. 2.94 kg of glycerol was then added to the combined mixture while continuing to mix to yield a uniform solution. The solution was cooled while continuing to stir at the same speed to yield a final topical formulation.

### Example 3: Ex Vivo/In Vitro Permeability Studies

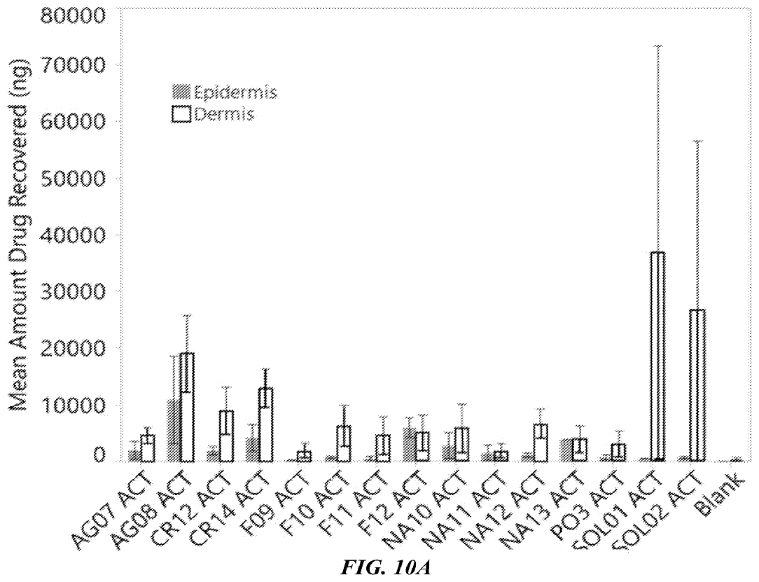

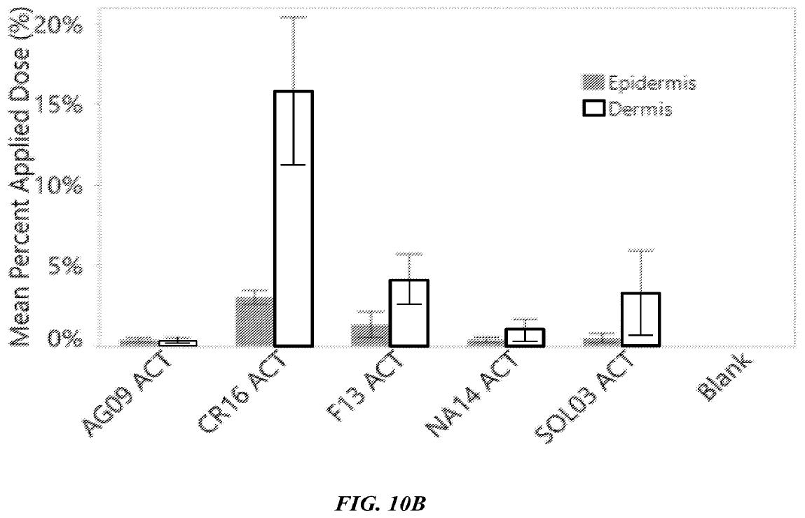

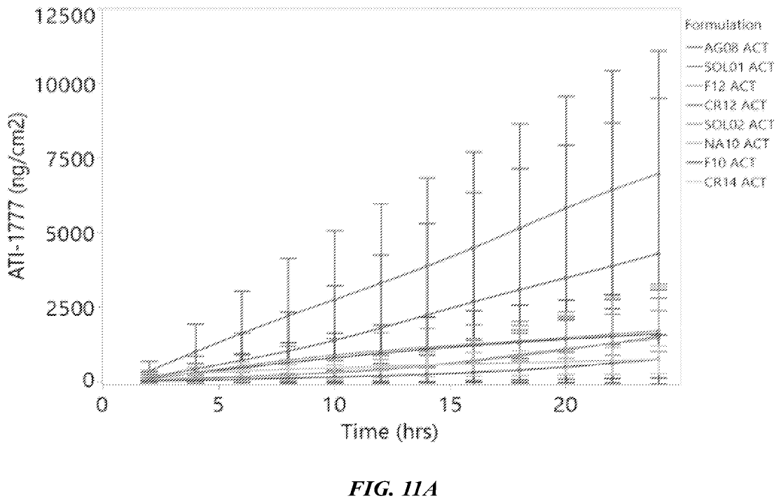

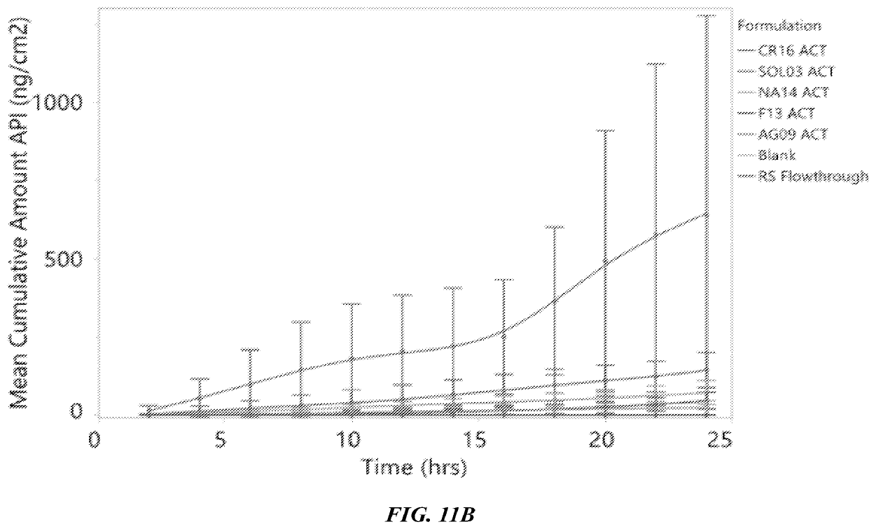

For the ex vivo permeability study, 10 mg of formulations 1-12 were applied to a test layer of skin as described above. Table 2 below represents data collected from the studies of various embodiments of the formulations disclosed herein (Formulations 1-3, 4 and 8 described above). The amount applied to the skin is reported in mg and represents the amount of Compound I in the formulation applied to the skin. FIGS. 10A and 10B compare the permeation of a formulation of Formulation 1 (SOL01), Formulation 2 (SOL02), and Formulation 3 (SOL03) into the dermis and the epidermis to various other Compound I containing formulations as percent of the applied dose. Specific formulations included in FIGS. 10a and 10b are an ointment formulation (PO3), non-aqueous gels (NA 10, 11, 12, 13, and 14), aqueous gels (AG07, 08, and 09), creams (CR12, CR14, CR16), and foams (F09, 10, 11, 12, and 13). FIGS. 11A and 11B compare the concentration of Compound I in the receptor solution of Formulation 1 (SOL01), Formulation 2 (SOL02), Formulation 3 (SOL03), Formulation 4 (SOL04), and Formulation 8 (SOL14) to various aqueous and non-aqueous gels, cream, and foam formulations.

TABLE 2

| | | | Average | | Average | Average |
|---|---|---|---|---|---|---|
| | Amount | Average | Epidermis | Average | Dermis | Amount in |
| | Applied | Epidermis | Amount (% | Dermis | Amount (% | Receptor |
| Formulation | to Skin | Amount | of applied | Amount | of applied | Solution |
| No. | (mg/cm²) | (ng) | dose) | (ng) | dose) | (ng) |
| 1 | 10 | 591 | 0.22% | 36864 | 15.03% | 4279 |
| 2 | 10 | 705 | 0.29% | 26738 | 10.66% | 1870 |
| 3 | 10 | 1141 | 0.51% | 7362 | 3.31% | 144 |
| 4 | 10 | 3126 | 1.4% | 32492 | 15% | 77.7 |
| 8 | 10 | 3614 | 1.7% | 20274 | 9.4% | 449.5 |

### Example 4: Phase 2a Study to Determine the Safety, Tolerability, Pharmacokinetics, and Efficacy of Topical JAK Inhibitor for Treatment of Moderate or Severe Atopic Dermatitis

Compound I is an inhibitor of JAK1/3 enzymes and has shown topical activity in skin inflammation models. Compound I was designed to be pharmacologically active in the skin with rapid systemic clearance to limit systemic exposure.

### Brief Method

Adult subjects with moderate or severe atopic dermatitis (AD) were randomized to Compound I topical solution 2.0% or vehicle treatment administered twice daily (BID). The primary endpoint was percent (%) change in mEASI score at Day 28. Other efficacy endpoints included IGA, BSA, PP-NRS. Safety assessments included Adverse Event (AE) assessments, vital signs, ECGs and laboratory evaluations. PK blood draws assessed systemic exposure.

### Detailed Method

Fifty (50) subjects, aged 18-65 years, male and female were selected to use either a topical formulation comprising 2% w/w Compound I or a placebo. Pertinent inclusion criteria for the study included: (i) a diagnosis of atopic dermatitis (AD) fulfilled the specified diagnostic criteria of Hanifin and Rajka (Hanifin J M, Rajka G. Diagnostic features of atopic dermatitis. Acta Derm Venereol. Suppl 1980; 92: 44-7, which is hereby incorporated by reference in its entirety); (ii) a 6-month history of AD; (iii) no significant AD flares for 4 weeks prior; (iv) at least 1 lesion measuring 3 cm³, but not located on the hands, feet, or genitalia; (v) a stable diagnosis of moderate or severe (Investigator's Global Assessment (IGA) score of 3 or 4); (vi) Eczema Area and Severity Index (EASI) score of ≤48; and (vii) have AD affecting 3% to 20% of Body Surface Area (BSA) (not including head, palms of feet, soles of feet, groin, and genitalia).

The 50 subjects were randomized into 2% Compound I treatment (n=23) and vehicle treatment (n=25) groups. Two of the subjects were randomized but not treated. Of the allocated subjects, 91% (n=21) of the Compound I treatment group and 72% (n=18) completed the study. The demographics and baseline characteristics of the study subjects is provided below in Table 3.

TABLE 3
Demographics and Baseline Characteristics of Study Subjects

| | |
|---|---|
| 2% Compound I | Vehicle |
| N = 23 | N = 25 |

| | | | | |
|---|---|---|---|---|
| Age Mean (SD) | 43.1 | (13.99) | 41.4 | (14.31) |
| Sex Male N(%) | 7 | (30.4) | 5 | (20.0) |
| Sex Female N(%) | 16 | (69.6) | 20 | (80.0) |
| Race | | | | |
| White | 15 | (65.2) | 12 | (48.0) |
| African American | 7 | (30.4) | 10 | (40.0) |
| Other | 1 | (4.3) | 3 | (12.0) |
| Disease Severity | | | | |
| Moderate | 22 | (95.7) | 24 | (96.0) |
| Severe | 1 | (4.3) | 1 | (4.0) |
| Baseline BSA | 9.61 | (5.433) | 6.96 | (4.286) |
| Mean Baseline EASI (SD) | 8.63 | (3.823) | 7.68 | (3.730) |

The topical formulation was applied topically twice per day, approximately 8 to 12 hours apart. Up to 8 mL, 1 mL at a time, is applied to an identified AD target area of the subject and rubbed into said area by the subject to achieve a thin film on the skin. Less than 8 mL may be used if less is required to fully cover the identified AD target area. Subjects document symptoms of AD, such as itching, on a daily basis. Subjects were asked to abstain from washing the treated area within 6 hours after application, minimize sun exposure, refrain from using moisturizers, emollients, and sunscreen on the treatment areas, and refrain from activities that would cause profuse sweating (e.g., exercise) for a period of 6 hours after each application. The study ran 4 weeks. On days 1, 8, and 15, a pharmacokinetic sample (e.g., blood) was collected from each subject about 2 hours after application of the topical formulation. On day 28, the following pharmacokinetic samples were collected:
Subgroup 1: One sample before application and one sample at 0.5 to 2.5 hours post application
Subgroup 2: One sample at 2.5 to 5 hours post application and one sample at least 2 hours later
Subgroup 3: One sample at 5 to 8 hours post application and one sample at least 2 hours later (prior to the subsequent application)

On each of these days 1, 8, 15, and 28, symptoms of AD in each subject were evaluated by EASI score, IGA, PP-NRS, and BSA. Primary efficacy of treatment was measured by a percent change from baseline EASI (EASI at day 1) to EASI at day 28.

Further measurements of efficacy included percent change in EASI between each of days 1, 8, 15, and 28 as well as change from baseline and/or prior evaluation of IGA, AD BSA, and PP-NRS on days 8, 15, and 28; percent of subjects achieving IGA scores of 0 to 1 combined with an improvement of 2 or more points from baseline by day 28; proportion of subjects who achieve 50%, 75%, and 90% improvement in EASI score (compared to baseline).

### Brief Results

50 subjects were enrolled in the study, of whom 48 (96.0%) met Full Analysis Set criteria. 39/48 (81.3%) subjects completed the study: 7(14.6%) vehicle subjects discontinued including all 3 subjects who discontinued due to AEs. Compound I was superior to vehicle with a 74.4% reduction in mean mEASI at week 4 compared to 41.4% in the vehicle group (1-sided p<0.001). Positive trends in favor of Compound I were observed in secondary efficacy endpoints, including mEASI 50/75/90 and IGA responders, and change in IGA, BSA and itch scores. Greater than 86% of the plasma samples exhibited Compound I levels below the lower limit of quantitation of 0.1 ng/mL. Mean drug levels were not greater than 5% of the IC50 of Compound I. Compound I was generally well tolerated with similar proportions of subjects reporting AEs in each treatment group (9/23 in the Compound I group and 9/25 in the vehicle group). No deaths or SAEs were reported. Most AEs were mild or moderate in severity. Two unrelated severe events (AF and elevated CPK) were reported in the Compound I arm.

### Detailed Results

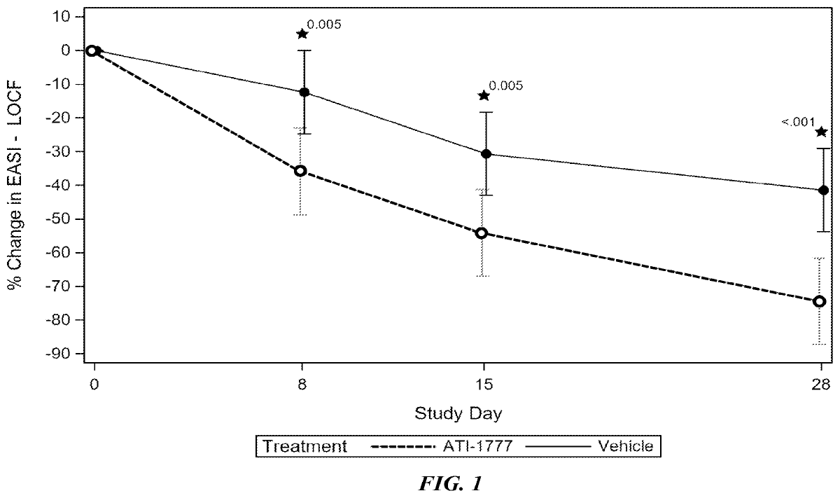

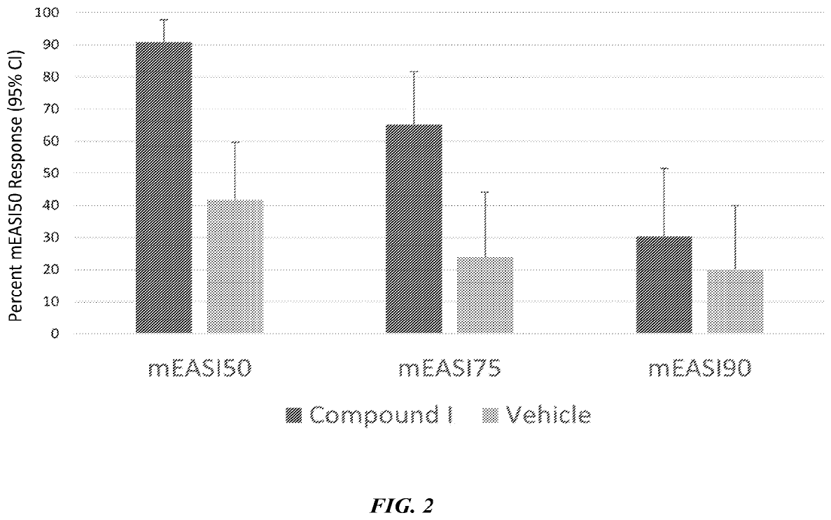

FIG. 1 shows the percent change in EASI score from baseline for the Compound I and vehicle treated subjects at days 8, 15, and 28. The data shows a significant greater improvement in EASI score from baseline versus vehicle, demonstrating definitive evidence of efficacy on a validated and recognized endpoint in AD.

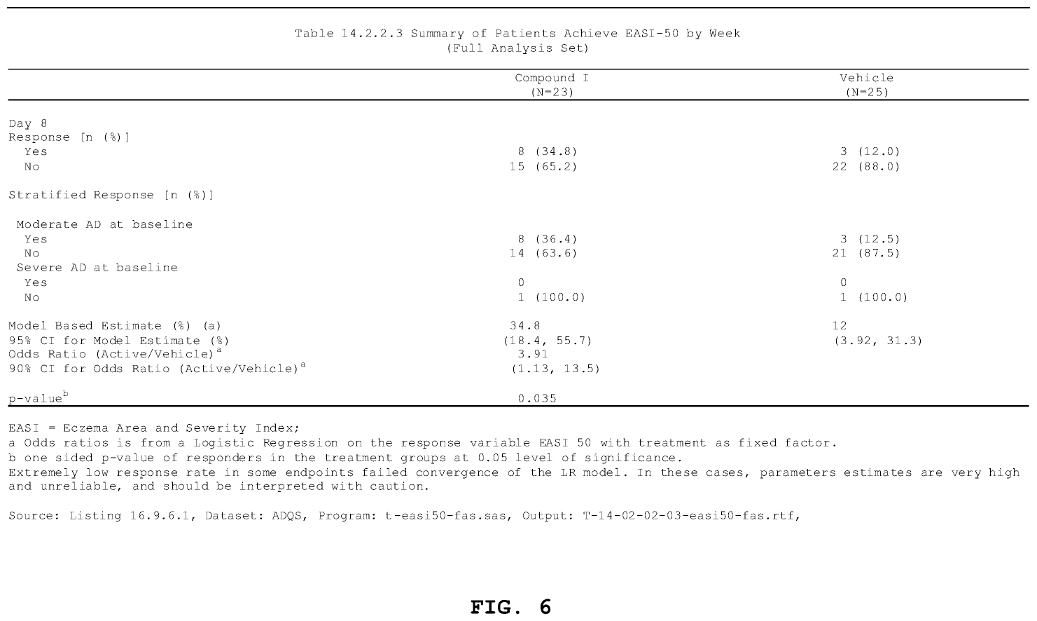

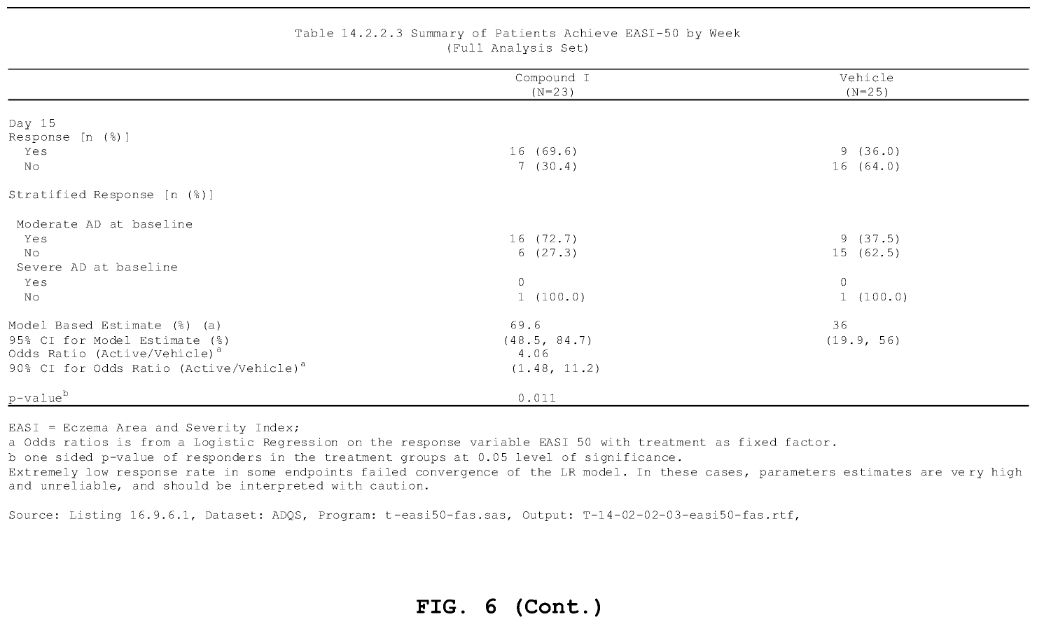

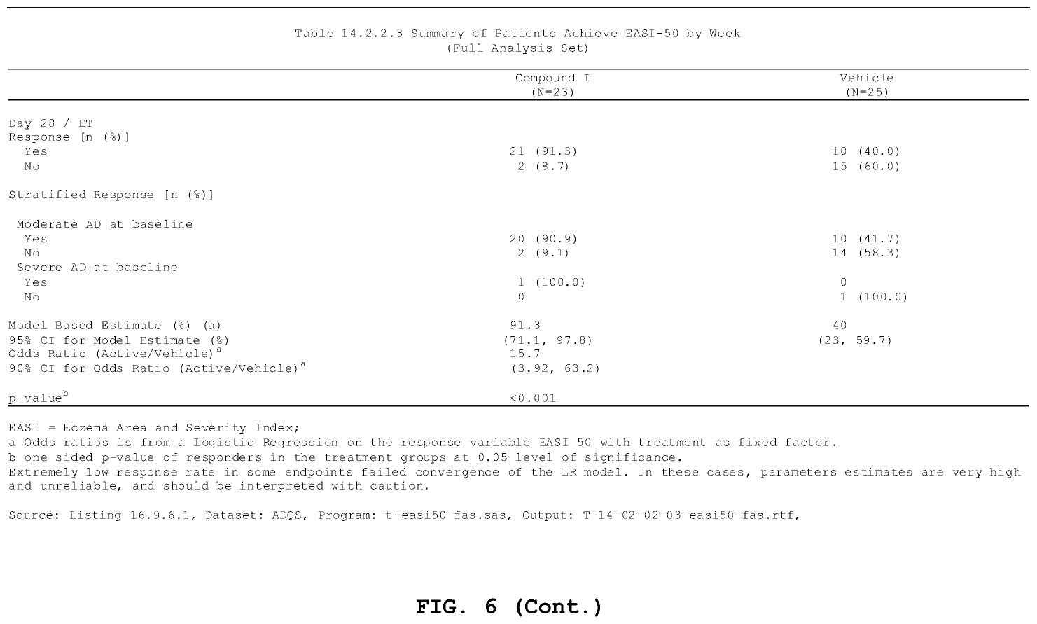

FIG. 6 is a table summarizing the EASI 50 responder data over the course of the study. As shown, at the end of week 1 (day 8), 34.8% of Compound I treated patients had achieved a 50% improvement in the EASI score compared to 12% of vehicle treated patients. At the end of week 2 (day 15), 69.6% of Compound I treated patients vs. 36% of vehicle treated patients achieved 50% improvement, and at the end of week 4 (day 28), 91.3% of Compound I treated patients vs. 40% of vehicle treated patients achieved 50% improvement in the EASI score (see also FIG. 2, a graph showing the proportion EASI 50, EASI 75 and EASI 90 responders in the Compound I and vehicle treated patients at day 28 of the study).

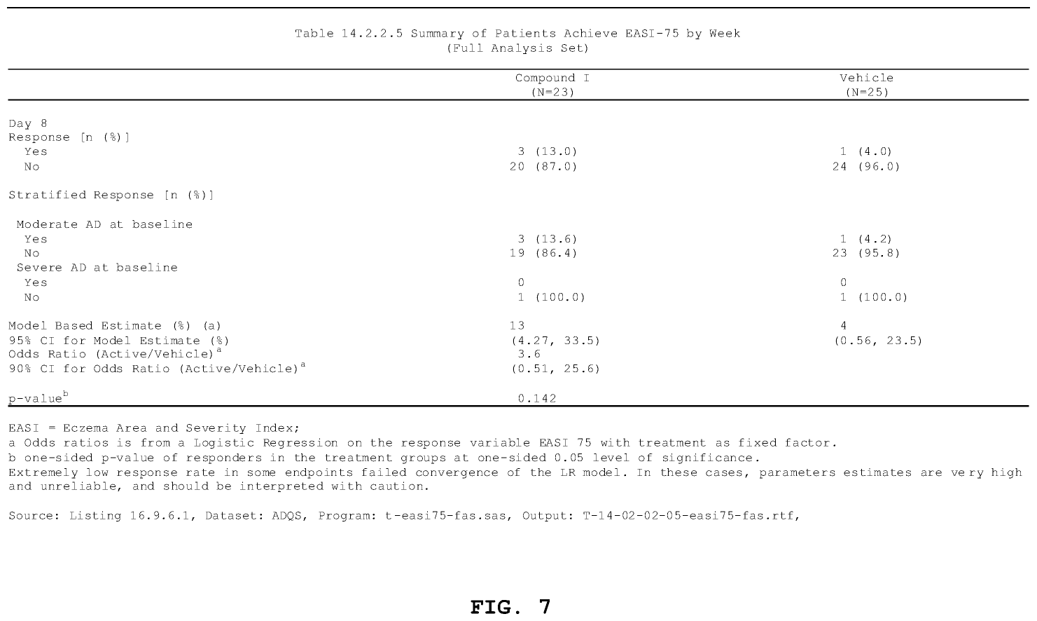

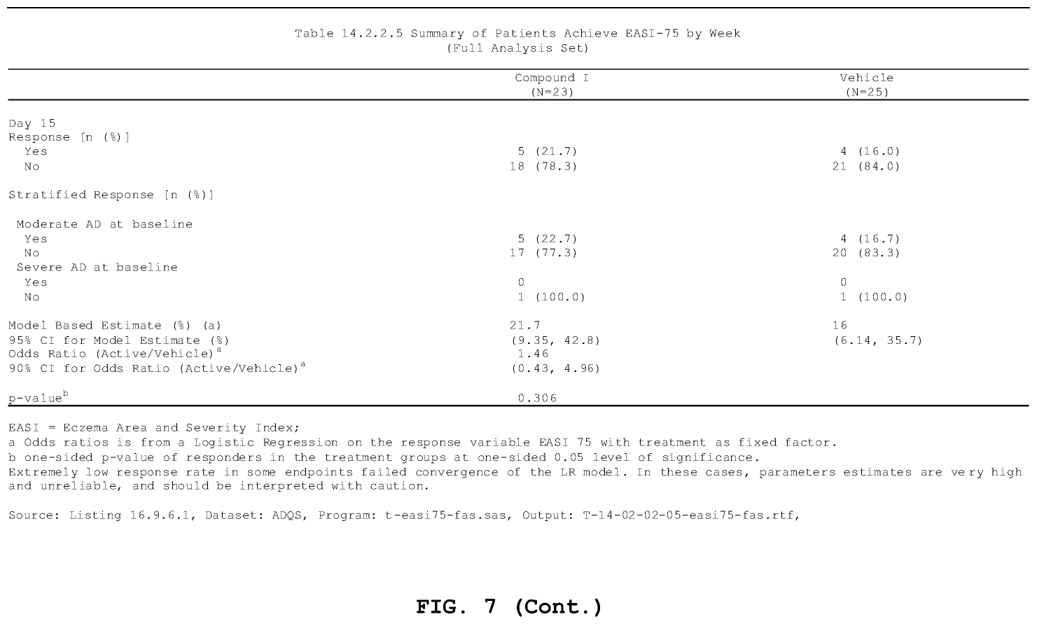

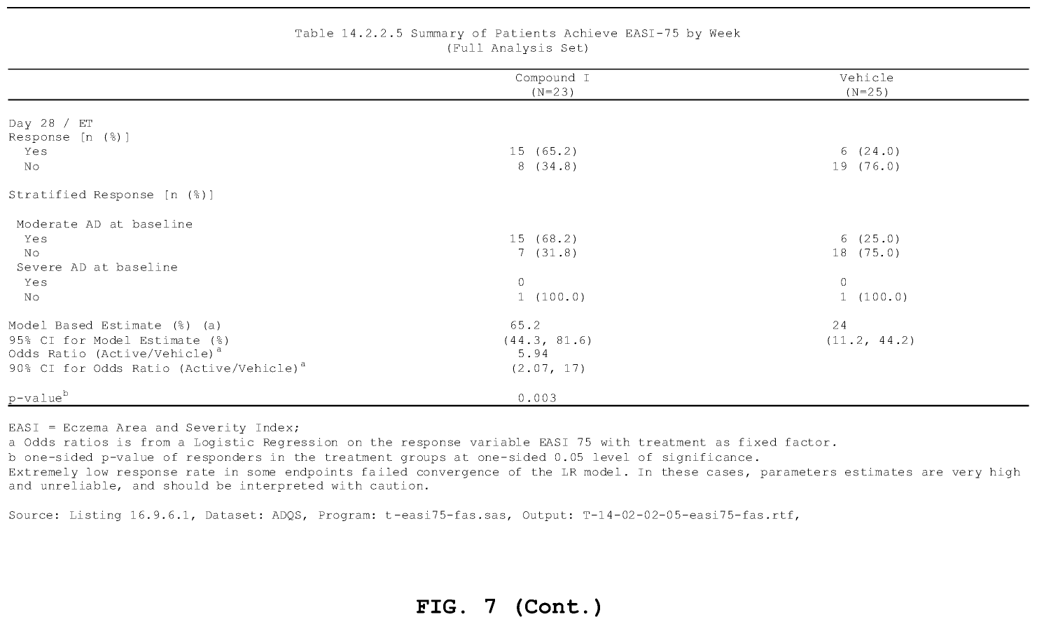

FIG. 7 is a table summarizing the EASI 75 responder data over the course of the study. As shown, at the end of week 1 (day 8) 13.0% of Compound I treated patients had achieved a 75% improvement in the EASI score compared to 4% of vehicle treated patients. At the end of week 2 (day 15), 21.7% of Compound I treated patients vs. 16% of vehicle treated patients achieved 75% improvement, and at the end of week 4 (day 28), 65.2% of Compound I treated patients vs. 24% of vehicle treated patients achieved 75% improvement in the EASI score (see also FIG. 2).

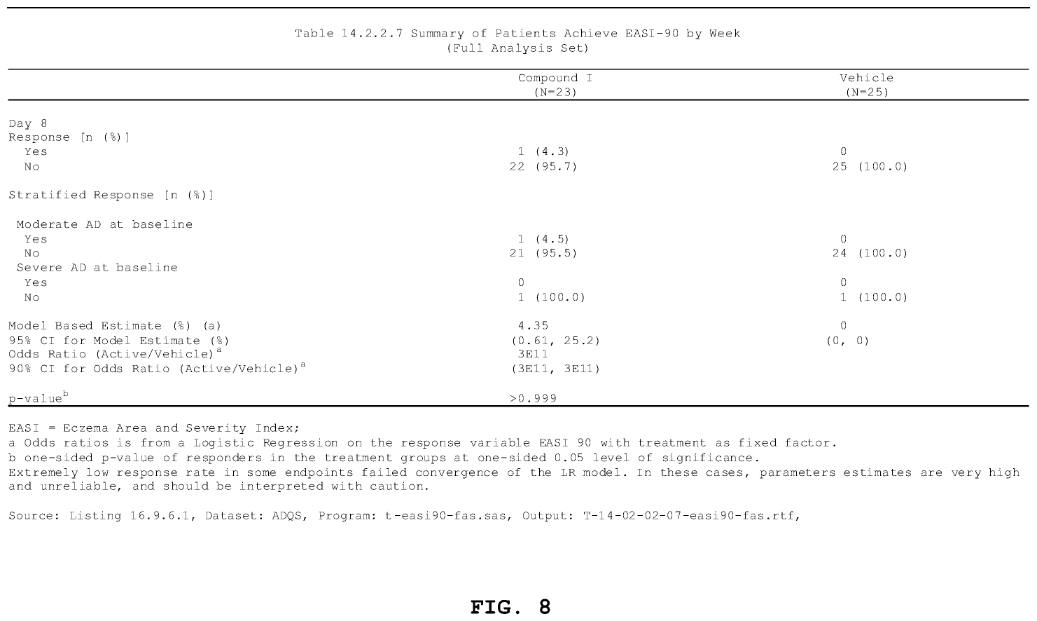

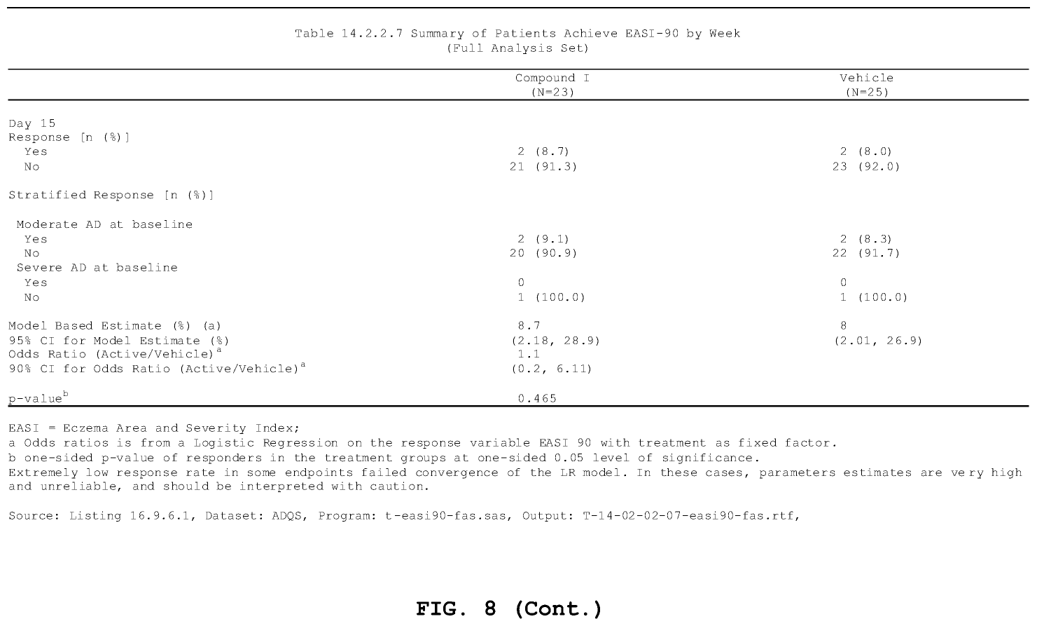

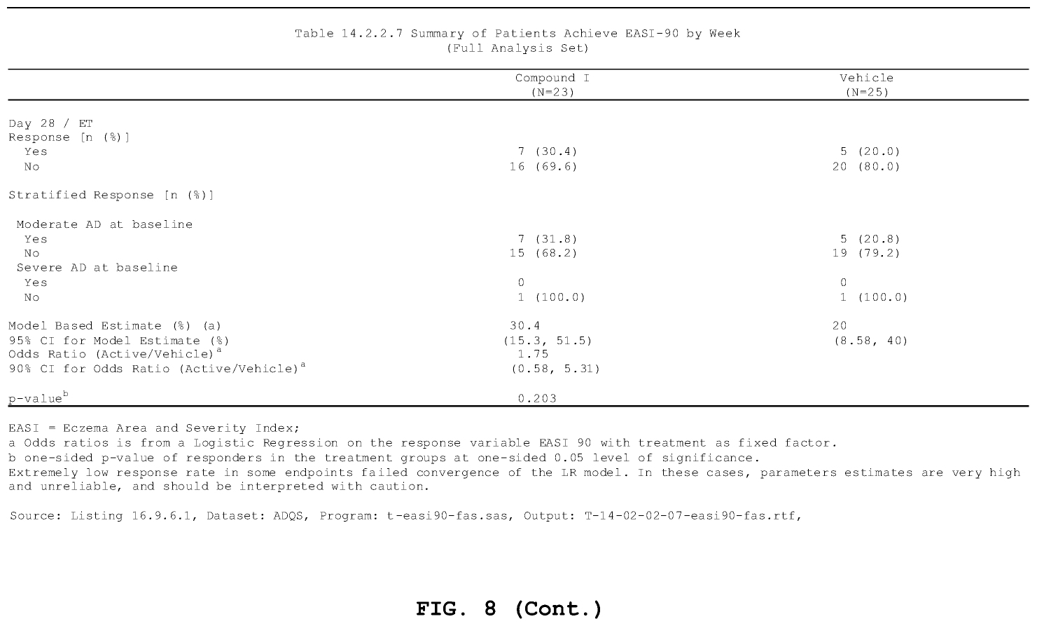

FIG. 8 is a table summarizing the EASI 90 responder data over the course of the study. As shown, at the end of week 1 (day 8) 4.3% of Compound I treated patients had achieved a 90% improvement in the EASI score compared to 0% of vehicle treated patients. At the end of week 2 (day 15), 8.7% of Compound I treated patients vs. 8% of vehicle treated patients achieved 90% improvement, and at the end of week 4 (day 28), 30.4% of Compound I treated patients vs. 20% of vehicle treated patients achieved 90% improvement in the EASI score (see also FIG. 2).

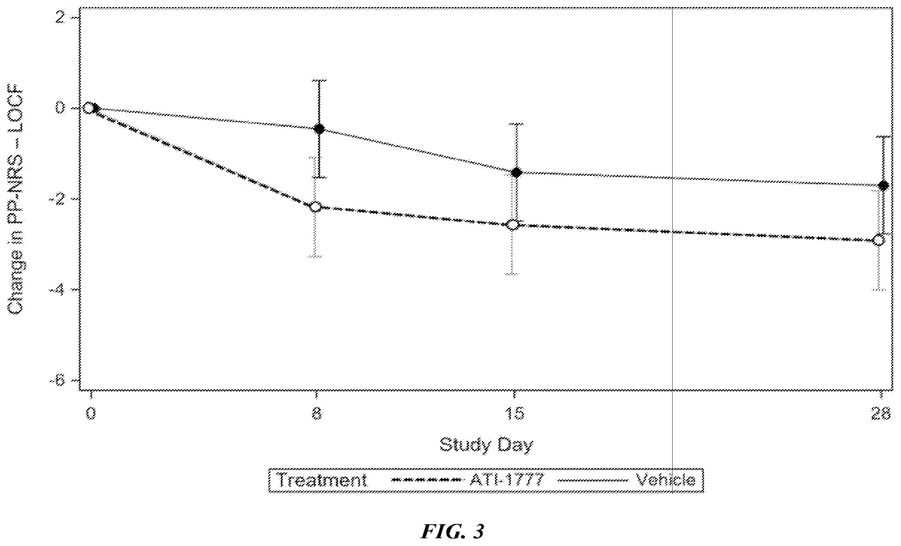

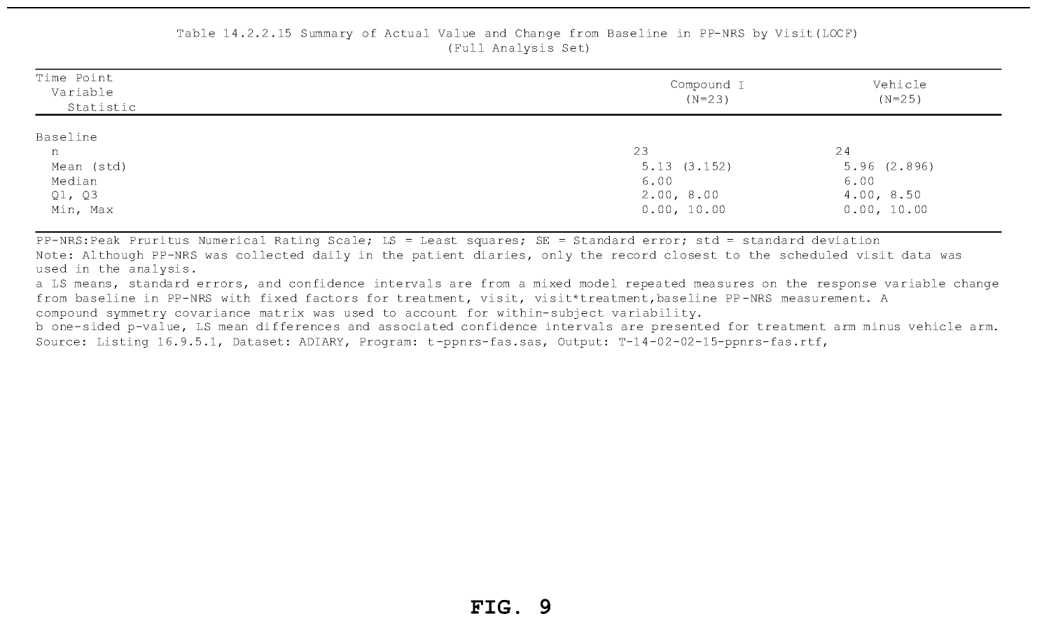

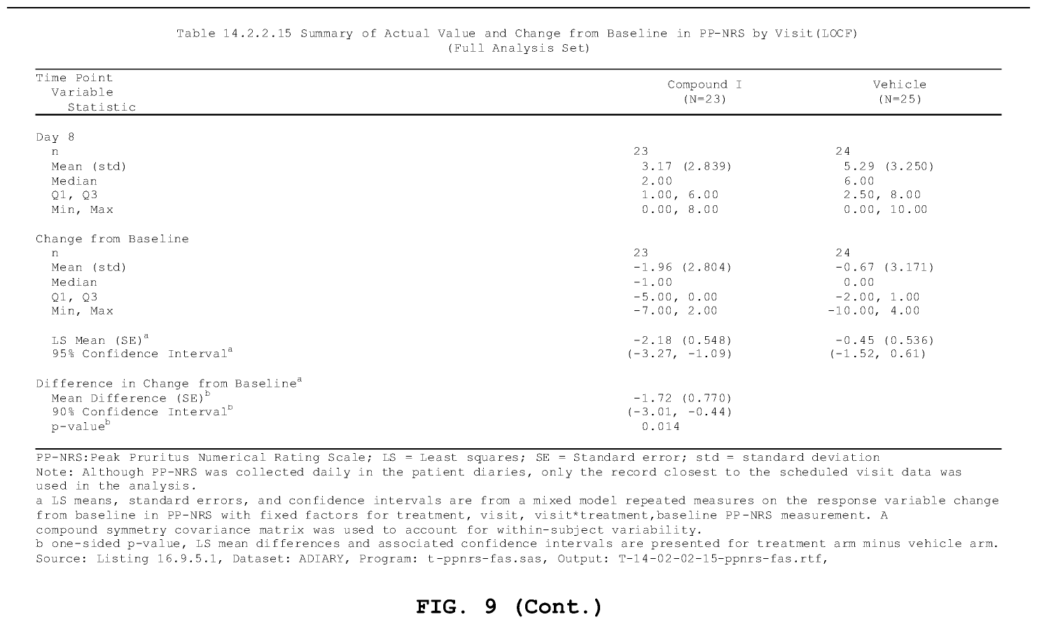

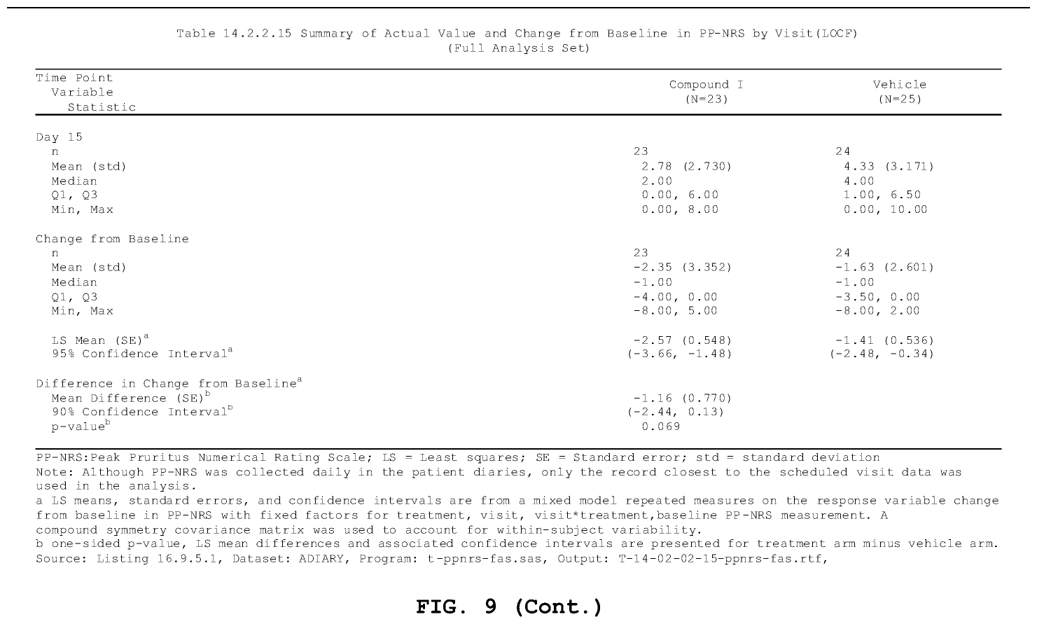

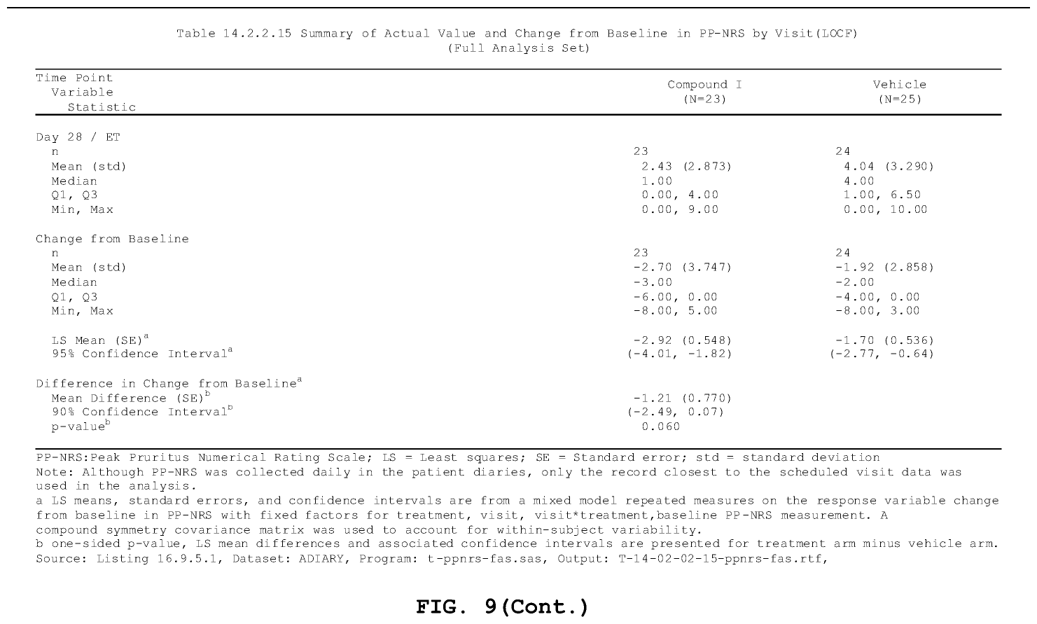

FIG. 9 is a table summarizing the change in baseline in Peak Pruritus Numerical Rating Scale (PP-NRS) by week over the course of the study and FIG. 3 shows a graph of this change. It is notable that after only one week, patients treated with Compound I experienced a mean decrease in their PP-NRS score of 1.72 relative to baseline. A 1.0 difference is considered a clinically meaningful reduction.

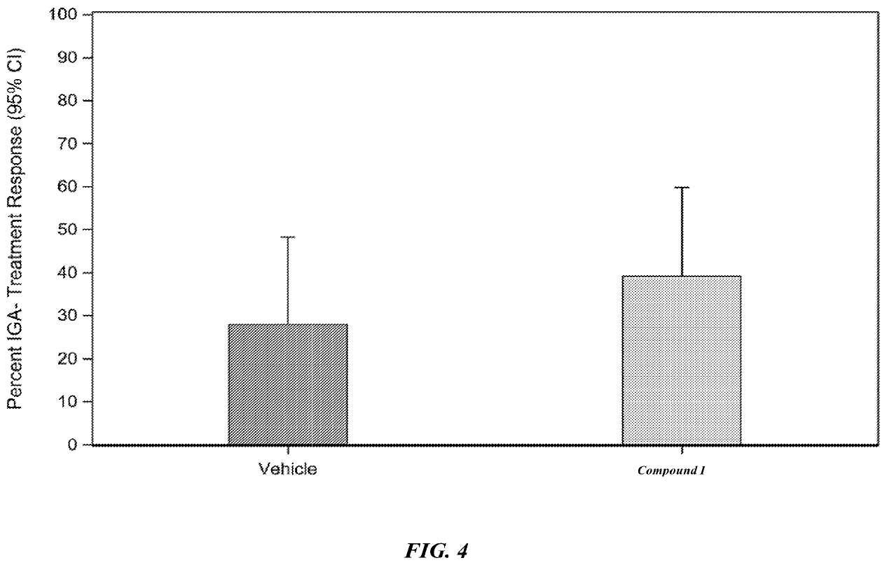

FIG. 4 shows the proportion of subjects (Compound I and vehicle treated) having an Investigator's Global Assessment (IGA) treatment response of 0 or 1 with ≥2 point improvement during the study.

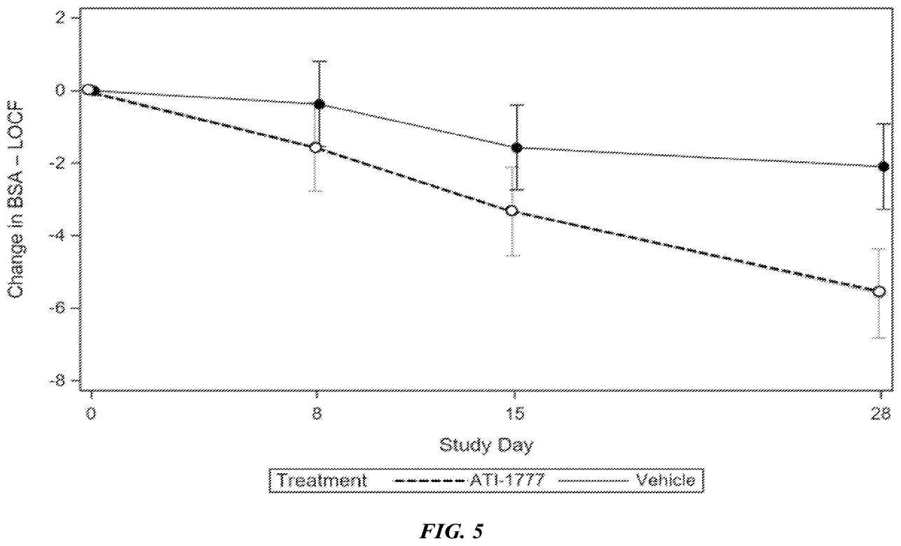

FIG. 5 shows the change in BSA impacted by the disease in Compound I and vehicle treated subjects from baseline at each of days 1, 8, 15 and 28.

As expected, topical administration of Compound I to patients in the study did not result in clinically relevant plasma concentration of Compound I in any of the patients. All plasma concentrations were less than half of the IC₅₀ (IC₅₀=36 ng/mL). The average plasma concentration in subjects receiving Compound I solution at days 1, 8 and 28 was as follows:
Day 1, 2 hr post dose: 1.604 ng/mL
Day 8, 2 hr post dose: 0.519 ng/mL
Day 28, pre dose: 0.328 ng/mL
   Only 3 subjects (6 total samples) with concentrations > 1/10^{th} the IC₅₀. No subject had a concentration of Compound I above 15 ng/mL. Lower limit of quantitation (LLOQ) was 0.100 ng/mL.

Tables 4 and 5 below show PK data for subjects exhibiting the lowest (Table 4) and highest (Table 5) plasma concentration of Compound I over the course of the study.

TABLE 4
Plasma Compound I Data (Low Exposure Examples)

| | | | | | | TIME | | | |
|---|---|---|---|---|---|---|---|---|---|
| TREATMENT | SUBJID | ANALYTE | VISIT | | HOUR | TEXT | | CONC | UNITS |
| Active | 004-0002 | Compound I | Day | 1 | 0 | Predose | | <LLOQ | ng/mL |
| | | | | | | | | (0.100) | |
| | 004-0002 | Compound I | Day | 1 | 2 | | | <LLOQ | ng/mL |
| | | | | | | | | (0.100) | |
| | 004-0002 | Compound I | Day | 8 | 0 | Predose | | 0.188 | ng/mL |
| | 004-0002 | Compound I | Day | 8 | 2 | | | 0.229 | ng/mL |
| | 004-0002 | Compound I | Day | 15 | 0 | Predose | | 0.100 | ng/mL |
| | 004-0002 | Compound I | Day | 15 | 2 | | | <LLOQ | ng/mL |
| | | | | | | | | (0.100) | |
| | 004-0002 | Compound I | Day | 28/ET | 0 | Predose | | 0.165 | ng/mL |
| | 004-0002 | Compound I | Day | 28/ET | 0.5 | 0.5-2.5 | hr | 0.168 | ng/mL |
| Active | 004-0004 | Compound I | Day | 1 | 0 | Predose | | <LLOQ | ng/mL |
| | | | | | | | | (0.100) | |
| | 004-0004 | Compound I | Day | 1 | 2 | | | <LLOQ | ng/mL |
| | | | | | | | | (0.100) | |
| | 004-0004 | Compound I | Day | 8 | 0 | Predose | | <LLOQ | ng/mL |
| | | | | | | | | (0.100) | |
| | 004-0004 | Compound I | Day | 8 | 2 | | | <LLOQ | ng/mL |
| | | | | | | | | (0.100) | |
| | 004-0004 | Compound I | Day | 15 | 0 | Predose | | <LLOQ | ng/mL |
| | | | | | | | | (0.100) | |
| | 004-0004 | Compound I | Day | 15 | 2 | | | <LLOQ | ng/mL |
| | | | | | | | | (0.100) | |
| | 004-0004 | Compound I | Day | 28/ET | 5 | 5-8 | hr | <LLOQ | ng/mL |
| | | | | | | | | (0.100) | |
| | 004-0004 | Compound I | Day | 28/ET | 7 | 7-10 | hr | 0.104 | ng/mL |
| Active | 004-0008 | Compound I | Day | 1 | 0 | Predose | | <LLOQ | ng/mL |
| | | | | | | | | (0.100) | |
| | 004-0008 | Compound I | Day | 1 | 2 | | | 1.62 | ng/mL |
| | 004-0008 | Compound I | Day | 8 | 0 | Predose | | 0.616 | ng/mL |
| | 004-0008 | Compound I | Day | 8 | 2 | | | 0.639 | ng/mL |
| | 004-0008 | Compound I | Day | 15 | 0 | Predose | | 0.740 | ng/mL |
| | 004-0008 | Compound I | Day | 15 | 2 | | | 0.512 | ng/mL |
| | 004-0008 | Compound I | Day | 28/ET | 2.5 | 2.5-5 | hr | 0.543 | ng/mL |
| | 004-0008 | Compound I | Day | 28/ET | 4.5 | 4.5-7 | hr | 0.441 | ng/mL |

TABLE 5
Plasma Compound I Data (High Exposure Examples)

| TREATMENT | SUBJID | ANALYTE | VISIT | | HOUR | TIME TEXT | | CONC | UNITS |
|---|---|---|---|---|---|---|---|---|---|
| Active | 012-0001 | Compound I | Day | 1 | 0 | Predose | | <LLOQ | ng/mL |
| | | | | | | | | (0.100) | |
| | 012-0001 | Compound I | Day | 1 | 2 | | | 14.9 | ng/mL |
| | 012-0001 | Compound I | Day | 8 | 0 | Predose | | 1.43 | ng/mL |
| | 012-0001 | Compound I | Day | 8 | 2 | | | 1.50 | ng/mL |
| | 012-0001 | Compound I | Day | 15 | 0 | Predose | | 0.521 | ng/mL |
| | 012-0001 | Compound I | Day | 15 | 2 | | | 1.05 | ng/mL |
| | 012-0001 | Compound I | Day | 28/ET | 0 | Predose | | 1.23 | ng/mL |
| | 012-0001 | Compound I | Day | 28/ET | 0.5 | 0.5-2.5 | hr | 3.71 | ng/mL |
| Active | 012-0017 | Compound I | Day | 1 | 0 | Predose | | <LLOQ | ng/mL |
| | | | | | | | | (0.100) | |
| | 012-0017 | Compound I | Day | 1 | 2 | | | 2.45 | ng/mL |
| | 012-0017 | Compound I | Day | 8 | 0 | Predose | | 0.845 | ng/mL |
| | 012-0017 | Compound I | Day | 8 | 2 | | | 0.459 | ng/mL |
| | 012-0017 | Compound I | Day | 15 | 0 | Predose | | 11.1 | ng/mL |
| | 012-0017 | Compound I | Day | 15 | 2 | | | 5.26 | ng/mL |
| | 012-0017 | Compound I | Day | 28/ET | 5 | 5-8 | hr | 0.536 | ng/mL |
| | 012-0017 | Compound I | Day | 28/ET | 7 | 7-10 | hr | 0.704 | ng/mL |
| Active | 13-0001 | Compound I | Day | 1 | 0 | Predose | | <LLOQ | ng/mL |
| | | | | | | | | (0.100) | |
| | 13-0001 | Compound I | Day | 1 | 2 | | | 12.9 | ng/mL |
| | 13-0001 | Compound I | Day | 8 | 0 | Predose | | 0.914 | ng/mL |
| | 13-0001 | Compound I | Day | 8 | 2 | | | 1.79 | ng/mL |
| | 13-0001 | Compound I | Day | 15 | 0 | Predose | | 0.598 | ng/mL |
| | 13-0001 | Compound I | Day | 15 | 2 | | | 1.86 | ng/mL |
| | 13-0001 | Compound I | Day | 28/ET | 0 | Predose | | 0.530 | ng/mL |
| | 13-0001 | Compound I | Day | 28/ET | 0.5 | 0.5-2.5 | hr | 3.99 | ng/mL |

Adverse events (AEs) of Compound I topical administration were monitored and recorded based on severity (e.g., mild, moderate, or severe) and causality. Overall safety of use of Compound I according to the methods disclosed herein is based on incidence of adverse events, subject vital signs, and physical examination results. Table 6 provides an overall summary of the number of subjects experiencing treatment emergent adverse events and severe adverse events, and Table 7 provides a summary of the adverse events reported by study participants. There were no serious adverse events reported and no patients in the Compound I group withdrew from the study due to an adverse event. Three patients in the vehicle group withdrew for COVID-19, SARS-CoV-2, and application site irritation.

TABLE 6
Overall Summary of Adverse Events (AEs)

| | 2% Compound I | Vehicle | Total |
|---|---|---|---|
| | N = 23 | N = 25 | N = 48 |
| Subjects with at least one | 9 (39.1) 16 | 9 (36.0) 10 | 18 (37.5) 26 |
| TEAE | | | |
| Subjects with at least one | 0 | 0 | 0 |
| SAE | | | |
| Subjects with at least one | 2 (8.7) 2 | 0 | 2 (4.2) 2 |
| severe TEAE | | | |
| Subjects with at least one | 1 (4.3) 1 | 1 (4.0) 1 | 2 (4.2) 2 |
| related TEAE | | | |
| Subjects with at least one | 0 | 3 (12.0) 3 | 3 (6.3) 3 |
| TEAE leading to | | | |
| discontinuation of study | | | |
| drug | | | |

TABLE 7
Adverse Events: Patients with at least one event

| | Compound I BID (N = 23) | | | Vehicle (N = 25) | | |
|---|---|---|---|---|---|---|
| | Mild | Moderate | Severe | Mild | Moderate | Severe |
| Preferred Term | 6 (26.1%) | 1 (4.3%) | 2 (8.7%) | 4 (16%) | 5 (20%) | 0 |
| Acrochordon | 1 (4.3) | 0 | 0 | 0 | 0 | 0 |
| Amylase increased | 1 (4.3) | 0 | 0 | 0 | 0 | 0 |
| Application site irritation | 0 | 0 | 0 | 0 | 1 (4.0) | 0 |
| Application site pruritus | 1 (4.3) | 0 | 0 | 0 | 0 | 0 |
| Atrial fibrillation | 0 | 0 | 1 (4.3) | 0 | 0 | 0 |
| Blood creatine | 1 (4.3) | 0 | 1 (4.3) | 0 | 0 | 0 |
| phosphokinase increased | | | | | | |
| Candida infection | 0 | 1 (4.3) | 0 | 0 | 0 | 0 |
| COVID-19 | 0 | 0 | 0 | 0 | 1 (4.0) | 0 |
| Dizziness | 1 (4.3) | 0 | 0 | 0 | 0 | 0 |
| Food poisoning | 1 (4.3) | 0 | 0 | 0 | 0 | 0 |
| Folliculitis | 1 (4.3) | 0 | 0 | 0 | 0 | 0 |
| Headache | 2 (8.7) | 0 | 0 | 0 | 0 | 0 |
| Lipase increased | 1 (4.3) | 0 | 0 | 0 | 0 | 0 |
| Oropharyngeal pain | 0 | 0 | 0 | 1 (4.0) | 0 | 0 |
| Pharyngitis streptococcal | 0 | 0 | 0 | 1 (4.0) | 0 | 0 |
| Rash | 0 | 0 | 0 | 1 (4.0) | 0 | 0 |
| SARS-CoV-2 test positive | 0 | 0 | 0 | 1 (4.0) | 0 | 0 |
| Sinus congestion | 0 | 0 | 0 | 0 | 1 (4.0) | 0 |
| Skin fragility | 0 | 0 | 0 | 0 | 1 (4.0) | 0 |
| Somnolence | 0 | 0 | 0 | 1 (4.0) | 0 | 0 |
| Tinea infection | 0 | 1 (4.3) | 0 | 0 | 0 | 0 |
| Transaminases increased | 1 (4.3) | 0 | 0 | 0 | 0 | 0 |
| Urinary tract infection | 0 | 1 (4.3) | 0 | 0 | 1 (4.0) | 0 |

### CONCLUSIONS

In this Phase 2a clinical trial, which consisted of a 4-week treatment period and a 2-week follow-up period during which no treatment was given, 50 subjects with moderate to severe AD were randomized in a 1:1 ratio into one of two arms: Compound I topical solution 2.0% w/w or vehicle applied twice daily. One of the key objectives of this first in human trial was to assess the “soft” aspect of this topical JAK inhibitor compound in subjects with moderate to severe atopic dermatitis. A preliminary analysis of pharmacokinetic plasma samples in the Compound I arm demonstrated that mean drug levels were never greater than 5% of the IC₅₀ of Compound I.

The primary efficacy endpoint of this trial was the percent change from baseline in the Eczema Area and Severity Index (EASI) score at week 4. The EASI was modified to exclude evaluation of the body areas that were not treated in the trial (i.e., the head, palms of hands, soles of feet, groin, or genitalia) (mEASI).

The Full Analysis Set (FAS), which was comprised of subjects randomized and administered at least one dose of trial medication, was used for the primary endpoint. Two subjects in the Compound I arm were excluded from the FAS analysis on the basis that they were lost to follow up after the baseline visit and did not have a formal record of being dosed. Key secondary efficacy endpoints, which were not powered for statistical significance, included the proportion of subjects who achieved 50% in EASI score (EASI-50) within 4 weeks of the start of treatment, the change from baseline in the Investigator's Global Assessment (IGA) score at each trial visit, change from baseline in Body Surface Area (BSA) at each trial visit, and change from baseline in peak pruritus numerical rating scale (PP-NRS) score over time. Only the primary efficacy endpoint was powered to detect a statistically significant outcome.

The FAS was comprised of 23 and 25 subjects who were randomized and had documented administration of at least one dose of Compound I or vehicle, respectively. The trial achieved its primary endpoint with a high degree of statistical significance (p<0.001), which corresponded to a 74.5% reduction in mEASI score from baseline at week 4 in subjects applying Compound I compared to a 41.4% reduction in subjects applying vehicle. In addition, a post-hoc analysis which included the two randomized subjects not in the FAS, using their baseline EASI score carried forward to day 28 as well as the pre-planned per protocol analysis of the percent change in EASI scores were both statistically significant.

In addition, positive trends in favor of Compound I were observed in key secondary efficacy endpoints, such as improvement in itch, percent of EASI-50 responders, and reduction in Body Surface Area impacted by disease.

Compound I was generally well tolerated. Nine subjects in each arm reported adverse events (9/23 and 9/25 in the Compound I and vehicle arm, respectively). No serious adverse events were reported. The most common adverse events (reported in ≥2 or more subjects in the Compound I arm) were increased blood creatinine phosphokinase and headache which were determined by the clinical trial investigators to be unrelated to Compound I. In the FAS analysis, two subjects from the Compound I arm withdrew from the trial (1 lost to follow up, 1 withdrew consent), while seven subjects withdrew from the vehicle arm (3 due to AEs and 4 withdrew consent). There were no reports of thrombosis.

BID treatment with Compound I topical solution was effective in adults with moderate or severe AD. Topically applied drug resulted in systemic plasma concentrations substantially below the IC50 and therefore not likely to have clinical significance. BID treatment with Compound I topical solution was generally well tolerated over 4 weeks of treatment. This study supports progression to Phase 2b studies.

### Example 5: Phase 2b Study to Determine the Safety, Tolerability, Pharmacokinetics, and Efficacy of Topical JAK Inhibitor for Treatment of Moderate or Severe Atopic Dermatitis (AD).

### Objectives and Endpoints

The primary objective of this study will be to assess the efficacy of Compound I solution in patients with moderate to severe AD following twice daily (BID) or once daily (QD) applications. The primary efficacy endpoint for this study will be the percent change from baseline in EASI score at week 4.

The secondary objectives of this study will be to further characterize the efficacy of the Compound I solution by assessing several additional endpoints. These endpoints will include (i) determining the percent change from baseline in Eczema Area and Severity Index (EASI) score at each study visit, (ii) determining the proportion of patients achieving Investigator's Global Assessment (IGA) Treatment Success (IGA-TS) defined an (IGA) score of 0 to 1 combined with an improvement of 2 or more points from baseline within 4 weeks of the start of treatment, (iii) determining the proportion of patients who achieve 50%, 75%, and 90% improvement in EASI score (EASI-50, EASI-75, and EASI-90, respectively) at each visit. Additionally, several change from baseline endpoint measurements will be assessed. These include (iv) change from Baseline in IGA score at each study visit, (v) change from Baseline in body surface area (BSA) at each study visit, (vi) change from Baseline in Peak Pruritus Numerical Rating Scale (PP-NRS) score over time, (vii) the change in the percent of BSA from Baseline to Day 28, (viii) the change in the PGIC from Baseline to Day 28, (ix) the proportion of patients with a score of either 1 or 2 on the PGIC from Baseline to Day 28, and (x) the change in the PP-NRS from Baseline to Day 28. Finally, the following additional secondary endpoints will also be assessed: (xi) the proportion of patients with a 4-point improvement in the PP-NRS at Day 28, (xii) assess Compound I trough Pharmacokinetics (PK) concentrations at each visit, and (xiii) safety and tolerability will be assessed by monitoring the frequency, duration, and severity of adverse events (AEs).

In addition to the above, the study will also investigate the following exploratory objectives: (i) assess the time to IGA score of “Clear” (0) or “Almost Clear” (1), (ii) assess the time to 50, 75, 90% improvement in the EASI, and (iii) assess the time to achieve PP-NRS score improvement of at least 2, 3, or 4-points. Safety will be assessed via the following outcomes: (i) recording incidence of treatment-emergent AEs (TEAEs) and SAEs, (ii) tracking routine patient laboratory values, (iii) tracking patient vital signs, (iv) performing physical examinations of the patients, and (v) monitoring the patients via 12-lead ECG. In assessing the pharmacokinetics of the Compound I solution, a sparse sampling approach will be employed because it is not anticipated that clinically relevant plasma concentrations will be observed. PK samples will be drawn prior to study medication application on Days 1, 15, and 28.

### Study Design

This will be a randomized, double-blind, vehicle-controlled, parallel-group study to determine the safety, tolerability, pharmacokinetics, and efficacy of Compound I solution following twice-daily (BID) applications or once daily (QD) to target areas of patients with moderate to severe AD.

The study will consist of an up to 30-day screening period, a 4-week treatment period, and a 2-week follow-up period. The total duration of the study for patients remaining until their final follow-up assessment will be up to 42 days.

Patients will undergo screening evaluations to determine eligibility up to 30 days prior to randomization. Patients who meet all entry criteria will be randomized on Day 1. Study medication will be administered for 4 weeks with the final dose of study medication administered the morning of Day 28. Patients will attend clinic visits on Days 8 and 28 for safety, efficacy, and PK/PD assessments. Patients will return 2 weeks after the last dose of study medication for a Post-Treatment Follow-Up (PTFU) Visit to assess for safety. All assessments will be performed as detailed in the Schedule of Events (SOE).

Patients will undergo up to 30 days of screening, followed by 4 weeks of treatment and 2 weeks of PTFU.

The start of the study will be the date on which the first patient provides informed consent, and the end of the study will be the date of the last patient's last assessment.

It is planned to enroll approximately 240 patients. Patients will be randomized in a 2; 2; 2; 1; 2:1 of Compound I Topical Solution (0.5, 1.0, 2.0% w/w) BID (twice daily) or vehicle (topical vehicle solution containing no Compound I) BID or Compound I Topical Solution (2.0% w/w) QD (once daily) or vehicle QD.

### Target Population

Pertinent inclusion criteria for this study includes: patients that (i) have a diagnosis of AD fulfilling the specified diagnostic criteria of Hanifin and Rajka (Hanifin and Rajka 1980); (ii) have at least a 6-month history of AD prior to the Screening Visit, and no significant AD flares for the 4 weeks prior to the Screening Visit; (iii) have at least 1 lesion that measures at least 3 cm² at the Screening Visit and on Day 1 prior to the first dose of study medication, this lesion must be representative of the patient's disease state, but not located on the scalp, face, palms of hands, soles of feet, groin, or genitalia; (iv) have a diagnosis of moderate or severe (IGA score 3 or 4) AD at the Screening Visit; and (v) have AD affecting not less than 10% BSA (not including hair bearing scalp, palms of hands, soles of feet, groin, and genitalia) at the Screening Visit.

Pertinent exclusion criteria for this study includes: Patients with any of the following are excluded from the study; (i) unstable course of AD (spontaneously improving or rapidly deteriorating) based on the patient history or as determined by the investigator during the Screening Period; (ii) refractory AD (i.e., AD that required frequent hospitalizations and/or frequent intravenous treatment for skin infections within the year before the Screening Visit); (iii) AD of a severity (EASI >48) that the patient is not a candidate for a vehicle-controlled study; (iv) any signs or symptoms associated with AD therapy (e.g., history of anaphylaxis, hypersensitivity reactions, skin atrophy, striae, pigmentary changes) that, in the investigator's opinion, might impair evaluation of the AD or which exposes the patient to unacceptable risk by study participation; (v) concomitant skin disease or clinically infected AD or presence of other skin disease in the area to be dosed that may interfere with study assessments; (vi) use of any of the following treatments within the indicated washout period prior to Day 1:
Phototherapy (ultraviolet A, ultraviolet B, or psoralen and ultraviolet A therapy) within 4 weeks prior to Day 1.
Systemic biologic immunosuppressant or immunomodulatory therapy (e.g., etanercept, alefacept, infliximab, dupilumab) within 12 weeks (or 5 half-lives of the product, whichever is longer) prior to Day 1.
Non-biologic immunosuppressants (e.g., methotrexate, retinoids, calcineurin inhibitors, cyclosporine, hydroxycarbamide [hydroxyurea], azathioprine) within 4 weeks prior to Day 1.
Janus kinase (JAK) inhibitors (systemic and topical) within 4 weeks prior to Day 1.
Systemic corticosteroids within 2 weeks prior to Day 1 (intranasal, inhaled, and topical ocular corticosteroids are allowed).
Cytostatic agents within 4 weeks prior to Day 1.
Crisaborole within 2 weeks prior to Day 1.
Systemic antibiotics within 30 days prior to Day 1.
Topical treatments for AD (corticosteroids, calcineurin inhibitors, topical H1 and H2 antihistamines, topical antimicrobials, and other medicated topical agents) within 2 weeks prior to Day 1.
Live attenuated vaccine treatment within 12 weeks prior to Day 1.
Other investigational product within 30 days or 5 half-lives (whichever is longer) prior to Day 1.

Safety Analysis: The FAS will be used for the analysis of safety data (AEs, clinical laboratory values, vital signs, examination results, and ECGs).

Plasma Drug Concentration: Plasma concentrations of Compound I will be summarized by nominal time point and day.

1. A method for treating a skin condition caused by immune deficiency in a human subject in need thereof, said method comprising: administering topically to a region of the subject affected by the skin condition, a composition comprising about 0.5% to about 4% of a compound having the structure of Compound I or a pharmaceutically acceptable derivative thereof

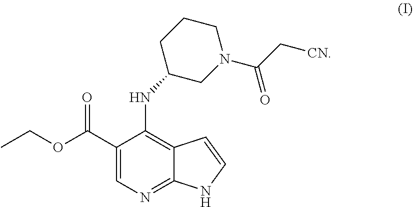 [SMILES: CCOC(=O)c1cnc2[nH]ccc2c1N[C@@H]1CCCN(C(=O)CC#N)C1]

2. The method of claim 1, wherein the skin condition is atopic dermatitis.

3. The method of claim 1, wherein said administering reduces one or more of an Investigator's Global Assessment (IGA) score, an Eczema Area and Severity Index (EAST) score, a Peak Pruritus Numerical Rating Scale (PP-NRS) score, or a Body Surface Area (BSA) measurement of the subject.

4. The method of claim 1, wherein the composition is administered topically to a skin lesion caused by the skin condition of the subject.

5. The method of claim 1, wherein the composition is administered once a day.

6. The method of claim 1, wherein the composition is administered twice a day.

7. The method of claim 1, wherein a composition comprising 2% w/w Compound I is administered twice daily.

8. The method of claim 1, wherein about 0.25 mL to about 8 mL of the composition comprising about 2% w/w of Compound I is administered topically to said subject.

9. The method of claim 1, wherein the composition is a cream, a gel, a gel-cream, a foam, or a solution.

10. The method of claim 1, wherein the composition is a solution.

11. The method of claim 1, wherein said administering comprises topical application of a solution comprising 2% w/w Compound I twice daily.

12. The method of claim 1 further comprising: administering one or more of a corticosteroid, calcineurin inhibitor, a phosphodiesterase-4 inhibitor, dupilumab, and a humanized monoclonal antibody that blocks signaling of interleukin (IL)-4/IL-13 to the subject.

13. A method for reducing severity and extent of skin lesions caused by an immune deficiency, said method comprising: administering topically to a human subject having the immune deficiency a composition comprising about 0.5% to about 4% of a compound having the structure of Compound I or a pharmaceutically acceptable derivative thereof

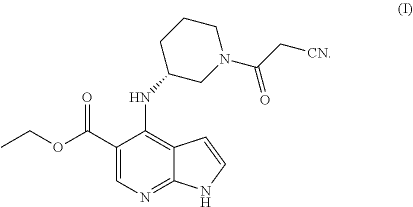 [SMILES: CCOC(=O)c1cnc2[nH]ccc2c1N[C@@H]1CCCN(C(=O)CC#N)C1]

14. The method of claim 13, wherein subject has been diagnosed with atopic dermatitis.

15. The method of claim 13, wherein the composition is administered once a day.

16. The method of claim 13, wherein the composition is administered twice a day.

17. The method of claim 13, wherein said administering reduces one or more of an IGA score, an EASI score, a PP-NRS score, or a BSA measurement of the subject.

18. The method of claim 13, wherein a composition comprising 2% w/w Compound I is administered twice daily.

19. The method of claim 13, wherein the composition is a cream, a gel, a gel-cream, a foam, or a solution.

20. The method of claim 13, wherein the composition is a solution.

21. The method of claim 13, wherein said administering comprises topical application of a solution comprising 2% w/w Compound I twice daily.

22. The method of claim 13, wherein about 0.25 mL to about 8 mL of the composition comprising about 2% w/w of Compound I is administered topically to said subject.

23. A topical formulation comprising ethyl (R)-4-((1-(2-cyanoacetyl)piperidin-3-yl)amino)-1H-pyrrolo[2,3-b]pyridine-5-carboxylate or a pharmaceutically acceptable derivative, salt, or solvate thereof, and a vehicle.

24. The topical formulation of claim 23, wherein the ethyl (R)-4-((1-(2-cyanoacetyl)piperidin-3-yl)amino)-1H-pyrrolo[2,3-b]pyridine-5-carboxylate or a pharmaceutically acceptable derivative, salt, or solvate thereof is present in the topical formulation at a concentration of about 0.5% w/w.

25. The topical formulation of claim 23, wherein the ethyl (R)-4-((1-(2-cyanoacetyl)piperidin-3-yl)amino)-1H-pyrrolo[2,3-b]pyridine-5-carboxylate or a pharmaceutically acceptable derivative, salt, or solvate thereof is present in the topical formulation at a concentration of about 2.0% w/w.

26. The topical formulation of claim 23, wherein the vehicle comprises a primary solvent and one or more co-solvents.

27. The topical formulation of claim 23, wherein the topical formulation further comprises an emollient.

28. The topical formulation of claim 23, wherein the topical formulation further comprises an antioxidant.
